(19) United States
(12) Patent Application Publication
(10) Pub. No.: US 2025/0144241 A1
(43) Pub. Date: May 8, 2025

(54) METHODS OF TARGETING REPETITIVE RNA IN HUNTINGTON'S DISEASE

(71) Applicant: The Regents of the University of California, Oakland, CA (US)

(72) Inventors: Eugene Yeo, La Jolla, CA (US); Kathryn H. Morelli, Encinitas, CA (US)

(21) Appl. No.: 18/837,257

(22) PCT Filed: Feb. 10, 2023
(86) PCT No.: PCT/US2023/062352

§ 371 Date: Aug. 9, 2024

Related U.S. Application Data
(60) Provisional application No. 63/309,330, filed on Feb. 11, 2022.

(51) Int. Cl. A61K 48/00; C12N 9/22; C12N 15/11; C12N 15/86

(52) U.S. Cl. CPC A61K 48/005; C12N 9/22; C12N 15/11; C12N 15/86; C12N 2310/20; C12N 2750/14143

(57) ABSTRACT

Provided herein are methods of treating Huntington's disease in a subject, the method comprising administering to the subject a therapeutically effective amount of a pharmaceutical composition comprising: (a) a guide RNA (gRNA), wherein the guide RNA comprises a repeat sequence complementary to a target CAG-expansion RNA, and wherein the gRNA allele-selectively targets the target CAG-expansion RNA; and (b) a CRISPR-associated protein or a nucleic acid sequence encoding the CRISPR-associated protein, wherein the CRISPR-associated protein comprises a Cas13d polypeptide.

### CROSS-REFERENCE TO RELATED APPLICATIONS

This application claims priority to U.S. Provisional Patent Application No. 63/309,330, filed on Feb. 11, 2022. The disclosure of the prior application is considered part of the disclosure of this application, and is incorporated herein by reference in its entirety.

### FEDERALLY SPONSORED RESEARCH OR DEVELOPMENT

This invention was made with Government support under Grant Nos. EY029166, NS103172, MH107367, AI132122, HG004659, and HG009889 awarded by the National Institutes of Health. The Government has certain rights in the invention.

### SEQUENCE LISTING

This application contains a Sequence Listing that has been submitted electronically as an ASCII text file named Sequence_Listing. The ASCII text file, created on Feb. 9, 2023, is 3,677 bytes in size. The material in the ASCII text file is hereby incorporated by reference in its entirety.

### BACKGROUND

Huntington's disease (HD) is a common autosomal dominant neurodegenerative disorder, caused by a CAG short tandem repeat (STR) expansion in exon 1 of the Huntingtin (HTT) gene. Onset of the motor symptoms, known as the clinical onset of HD, can occur from childhood to old age, with a mean age of onset at ˜45 years followed by inexorable disease progression. The therapies currently available to HD patients offer only moderate symptom relief, and the affected individuals typically die 15-20 years post-diagnosis due to complications.

Mutant HTT protein (mHTT) affects a variety of cellular functions. It binds and interacts with DNA in many genes, resulting in transcriptional dysregulation, neuronal dysfunction and, eventually, degeneration. Considering the pathogenic events that occur downstream of mHTT form a complex web, targeting of individual pathways is either too difficult to achieve cleanly or insufficient to modify the disease course of HD. A major focus of HD therapeutic development has recently shifted towards targeting the root of the disease, causative mutant HTT. Besides the toxicity of the mutated HTT protein, an increasing body of evidence indicates that mutant HTT mRNA also contributes to disease pathogenesis; consequently, strategies to suppress both HTT transcripts and protein levels would be most beneficial as a treatment. RNA interference (RNAi) and antisense oligonucleotide (ASO) strategies have shown preclinical efficacy and are being tested in clinical trials. However, most of these approaches do not precisely differentiate mutant HTT from the normal allele.

Most HD patients are heterozygous for the CAG expansion, and normal HTT plays important roles in brain development as well as the adult central nervous system (CNS). In the adult brain, HTT plays a part in intracellular vesicle trafficking, transcriptional regulation, and synaptic connectivity. While partial reduction of normal HTT levels is tolerable for a short period, long-term (years) ramifications of reductions are unclear given its involvement in a myriad of biological functions. Sustained reduction of normal HTT levels might exacerbate HD pathogenesis. The recent termination of the phase III clinical trial of an ASO (non-allele selective HTT-lowering) in HD patients underscores the urgency to develop strategies that can selectively and effectively suppress mutant HTT expression.

### SUMMARY

Provided herein are methods of treating Huntington's disease in a subject, the method comprising administering to the subject a therapeutically effective amount of a pharmaceutical composition comprising: (a) a guide RNA (gRNA), wherein the guide RNA comprises a repeat sequence complementary to a target CAG-expansion RNA, and wherein the gRNA allele-selectively targets the target CAG-expansion RNA; and (b) a CRISPR-associated protein or a nucleic acid sequence encoding the CRISPR-associated protein, wherein the CRISPR-associated protein comprises a Cas13d polypeptide.

In some embodiments, the repeat sequence of the guide RNA comprises GTC, TCG, or CGT. In some embodiments, the gRNA comprises a sequence selected from the group consisting of SEQ ID NO: 1, SEQ ID NO: 2, or SEQ ID NO: 3. In some embodiments, the gRNA further comprises a U6 promoter sequence.

In some embodiments, the target CAG-expansion RNA comprises more than 30 copies of a CAG repeat sequence. In some embodiments, the target CAG-expansion RNA comprises between 31 and 60 copies of a CAG repeat sequence. In some embodiments, the target CAG-expansion RNA comprises more than 60 copies of a CAG repeat sequence. In some embodiments, the target CAG-expansion RNA comprises more than 100 copies of a CAG repeat sequence. In some embodiments, the target CAG-expansion RNA comprises a mutant huntingtin gene (HTT).

In some embodiments, the Cas13d is Ruminococcus Flavefaciens XPD3002 (Rfx) Cas13d. In some embodiments, the Cas13d is tagged with a human influenza hemagglutinin (HA) epitope. In some embodiments, the nucleic acid sequence encoding the Cas13d polypeptide further comprises an RNA polymerase II promoter sequence or an RNA polymerase III promoter sequence. In some embodiments, the RNA polymerase III promoter sequence is an EF1a promoter sequence.

In some embodiments, the pharmaceutical composition comprises a vector, wherein the vector comprises at least one of (a) gRNA and (b) CRISPR-associated protein. In some embodiments, the vector comprises both (a) and (b). In some embodiments, the vector is a viral vector. In some embodiments, the viral vector is an adeno-associated viral vector (AAV), lentiviral vector, or an adenoviral vector.

In some embodiments, the subject is under 18 years old. In some embodiments, the subject is older than 18 years old. In some embodiments, the administration of the pharmaceutical composition comprises intrastriatal administration. In some embodiments, the administration of the pharmaceutical composition reduces mRNA and/or protein expression of the target CAG-expansion RNA.

Also provided herein are recombinant allele-selective expression systems for CRISPR/Cas-directed RNA targeting of a target CAG-expansion RNA comprising: (a) a guide RNA, wherein the guide RNA comprises a repeat sequence complementary to the target CAG-expansion RNA; and (b) a CRISPR-associated protein or a nucleic acid sequence encoding the CRISPR-associated protein, wherein the CRISPR-associated protein comprises a Cas13d polypeptide.

In some embodiments, the repeat sequence of the guide RNA comprises GTC, TCG, or CGT. In some embodiments, the guide RNA comprises a sequence selected from the group consisting of SEQ ID NO: 1, SEQ ID NO: 2, or SEQ ID NO: 3. In some embodiments, the gRNA further comprises a U6 promoter sequence.

In some embodiments, the target CAG-expansion RNA comprises more than 30 copies of a CAG repeat sequence. In some embodiments, the target CAG-expansion RNA comprises between 31 and 60 copies of a CAG repeat sequence. In some embodiments, the target CAG-expansion RNA comprises more than 60 copies of a CAG repeat sequence. In some embodiments, the target CAG-expansion RNA comprises more than 100 copies of a CAG repeat sequence. In some embodiments, the target CAG-expansion RNA comprises a mutant huntingtin gene (HTT).

In some embodiments, the Cas13d is Ruminococcus Flavefaciens XPD3002 (Rfx) Cas13d. In some embodiments, the Cas13d is tagged with an HA epitope. In some embodiments, the nucleic acid sequence encoding the Cas13d polypeptide further comprises an RNA polymerase II promoter sequence or an RNA polymerase III promoter sequence. In some embodiments, the RNA polymerase III promoter sequence is an EF1a promoter sequence.

In some embodiments, the recombinant expression system is delivered into a cell. In some embodiments, the cell is a mammalian cell. In some embodiments, the cell is derived from a subject diagnosed as having Huntington's disease.

In some embodiments, the recombinant expression system is comprised in a vector. In some embodiments, (a) and (b) are comprised within a same vector. In some embodiments, the vector is a viral vector. In some embodiments, the viral vector is an adeno-associated viral vector (AAV), lentiviral vector, or an adenoviral vector.

Also provided herein are vectors comprising a nucleic acid encoding (i) a guide RNA, wherein the guide RNA comprises a repeat sequence complementary to a target CAG-expansion RNA, and wherein the gRNA allele-selectively targets the target CAG-expansion RNA; and (ii) a CRISPR-associated protein, wherein the CRISPR-associated protein comprises a Cas13d polypeptide.

In some embodiments, the repeat sequence of the guide RNA comprises GTC, TCG, or CGT. In some embodiments, the gRNA comprises a sequence selected from the group consisting of SEQ ID NO: 1, SEQ ID NO: 2, or SEQ ID NO: 3. In some embodiments, the gRNA further comprises a U6 promoter sequence.

In some embodiments, the target CAG-expansion RNA comprises more than 30 copies of a CAG repeat sequence. In some embodiments, the target CAG-expansion RNA comprises between 31 and 60 copies of a CAG repeat sequence. In some embodiments, the target CAG-expansion RNA comprises more than 60 copies of a CAG repeat sequence. In some embodiments, the target CAG-expansion RNA comprises more than 100 copies of a CAG repeat sequence. In some embodiments, the target CAG-expansion RNA comprises a mutant huntingtin gene (HTT).

In some embodiments, the Cas13d is Ruminococcus Flavefaciens XPD3002 (Rfx) Cas13d. In some embodiments, the Cas13d is tagged with an HA epitope. In some embodiments, the nucleic acid sequence encoding the Cas13d polypeptide further comprises an RNA polymerase II promoter sequence or an RNA polymerase III promoter sequence. In some embodiments, the RNA polymerase III promoter sequence is an EF1a promoter sequence.

In some embodiments, the vector is delivered into a cell. In some embodiments, the cell is a mammalian cell. In some embodiments, the cell is derived from a subject diagnosed as having Huntington's disease. In some embodiments, the vector is a viral vector. In some embodiments, the viral vector is an adeno-associated viral vector (AAV), lentiviral vector, or an adenoviral vector.

Also provided herein are cells comprising any one of the recombinant allele-selective expression systems or any one of the vectors described herein.

Also provided herein are pharmaceutical compositions comprising any one of the cells described herein and a pharmaceutically acceptable carrier.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIGS. 1A-1G show development of an RNA-targeting, Cas13d-based gene therapy approach for HD. FIG. 1A shows a treatment scheme of a single gene therapy that expresses Cas13d and a gRNA designed to eliminate CAG-expanded HTT HTTRNA in both human striatal neuronal cultures derived from patient iPSCs and in the striatum of an established mouse model of HD, zQ175/+. FIG. 1B shows an exemplary diagram of a series of CAG-expanded, RNA-targeting vectors that consists of (1) Cas13d tagged with an HA epitope and (2) one of three U6 promoter-driven Rfx CRISPR-Cas13d gRNAs (denoted as CAGEX gRNA 1-3). FIG. 1C shows Western blot analysis results of polyQ protein from protein lysates isolated from HEK293 cells transfected with a CAG105 repeat plasmid and each candidate Cas13d vector. FIGS. 1D-1E show RNA dot blot analysis (FIG. 1D) and quantification (FIG. 1E) of CAG-expanded RNA within HEK293 cells transfected with a CAG105 repeat plasmid along with a nontargeting control (NT) or CAGEX-2 vector (one-way ANOVA, Tukey post hoc test, ****P<0.0001; n=1 technical replicates, n=3 biological replicates). FIGS. 1F-1G show RNA dot blot analysis (FIG. 1F) and quantification (FIG. 1G) of CUG-expanded RNA within HEK293 cells transfected with a CUG105 repeat plasmid along with a NT or CAGEX-2 vector (one-way ANOVA, Tukey post hoc test, n=3 technical replicates, n=3 biological replicates).

FIGS. 2A-2I show that Cas13d-CAG^{EX} reduces mHTT mRNA and protein in cells derived from patients with HD. FIG. 2A shows an exemplary schematic of the differentiation protocol used for iPSC-derived neurons treated with a lentiviral system expressing Cas13d-CAG^{EX}. FIG. 2B shows representative immunofluorescence images of cells at day 32 in control neuronal cells, showing that the cells are positive for striatal markers, DARPP-32 (top) and CTIP2 (bottom). Scale bar, 20 μm. n=1 technical replicate. FIGS. 2C-2D show RNA dot blot analysis (FIG. 2C) and quantification (FIG. 2D) of CAG-expanded RNA within MSN cells transduced with Cas13d-NT- or Cas13d-CAG^{EX}-expressing lentiviral vectors (oneway ANOVA, Tukey post hoc test, ***P<0.001). HD 66 Cas13d-CAG^{EX} P=0.00037, HD 77 Cas13d-CAG^{EX} P=0.00054, HD 109 Cas13d-CAG^{EX} P=0.00067; n=3 technical replicates, n=3 biological replicates. Data are presented as mean values±s.e.m. FIGS. 2E-2G show quantification of mHTT aggregates within MSN cells transduced with Cas13d-NT- or Cas13d-CAG^{EX}-expressing lentiviral vectors (FIG. 2E) (one-way ANOVA, Tukey post hoc test, ****P<0.0001, n=3 technical replicates, n=4 differentiations, 200 cells per experimental group). Representative images of the analysis are in FIG. 2F and FIG. 2G (scale bar, 20 μm). Data are presented as mean values±s.e.m. FIGS. 2H-2I show quantification of mutant and WT HTT RNA via allele-specific RT-qPCR in GM04723 (FIG. 2H) and GM02151 (FIG. 2I) fibroblasts (two-way ANOVA, Tukey post hoc test, ****P<0.0001, n=5 biological replicates). DAPI, 4,6-diamidino-2-phenylindole.

FIGS. 3A-3F show that Cas13d-CAG^{EX} partially reverses the molecular phenotypes of HD in patient iPSC-derived neurons. FIGS. 3A-3F show scatter plots of a common list of 988 upregulated and downregulated DEGs within HD 109, HD 77 and HD 66 treated with either Cas13d-NT (HD 109 (FIG. 3A), HD 77 (FIG. 3B), and HD 66 (FIG. 3C)) or Cas13d-CAG^{EX} (HD 109 (FIG. 3D), HD 77 (FIG. 3E), and HD 66 (FIG. 3F)) lentivirus vector compared to controls 1-3 referred to as ‘control’. The most significant HD-associated DEGs defined by a twofold change from control and a FDR-adjusted P<0.00001 are distinguished from total HD-associated DEGs with a broken vertical black line on the x axis (log 2 fold change) and a solid gray line on the y axis (−log₁₀ (P)), showing partial reversal of HD-mediated changes in the human transcriptome defined in FIGS. 3A-3C by Cas13d-CAG^{EX} in FIGS. 3D-3F.

FIGS. 4A-4H show therapeutic efficacy of Cas13d-CAG^{EX} in a full-length knock-in mouse model of HD. FIG. 4A shows an exemplary timeline of experimental design and outcome measures. FIGS. 4B-4E show motor function and body weight. Mice were tested on a 5-mm balance beam and time crossing the beam (traverse time) was recorded at the indicated ages in zQ175/+HD (FIG. 4B) or WT mice (FIG. 4C) with the indicated treatments. ***P=0.0004, **P=0.0057, two-way ANOVA with Bonferroni post hoc tests. Body weight in zQ175/+HD mice (FIG. 4D) or WT mice (FIG. 4E) with the indicated treatments (n=10, 5 males and 5 females per group). Data are presented as mean values±s.e.m. FIG. 4F shows representative MRI images of the zQ175/+HD and WT mice injected with the indicated AAVs. FIGS. 4G-4H show striatal (FIG. 4G) and neocortex (FIG. 4H) volume was quantified from 3D structural MRI in the indicated groups at 9 months of age (7 months after AAV injections). *P<0.05, ***P<0.001, one-way ANOVA with Tukey analysis. n=6 mice per group, 3 females and 3 males per group, 9 months of age. FIG. 4G shows P (Q175+Cas13d-NT versus Q175+Cas13d-CAG^{EX})=0.0196; P (Q175+Cas13d-NT versus WT+Cas13d-NT)=0.0241. FIG. 4H shows P (Q175+Cas13d-NT versus Q175+Cas13d-CAG^{EX})=0.0451; P (Q175+Cas13d-NT versus WT+Cas13d-NT)=0.0005.

FIGS. 5A-5K show allele-specific knockdown of mutant HTT protein and mRNA by Cas13d-CAG^{EX} in a full-length knock-in mouse model of HD. FIGS. 5A-5B show representative mHTT aggregates detected by immunostaining with EM48 antibody in zQ175/+ mice injected with Cas13d-NT (FIG. 5A) or Cas13d-CAG^{EX} (FIG. 5B). Scale bar, 10 μm. FIG. 5C shows quantification of mHTT aggregates in the zQ175 mice injected with AAV9-Cas13d-NT or AAV9-Cas13d-CAG^{EX}. *P<0.05 by two-sided Student's t-test. n=4 mice per group, 2 females and 2 males per group, 10 months of age. P=0.0261. FIG. 5D shows quantification of mutant and WT mRNA via allele-specific RT-qPCR in Q175/+ mice treated with either Cas13d-NT or Cas13d-CAG^{EX} (two-way ANOVA, Tukey post hoc test, ****P<0.0001, n=4 per experimental group). FIGS. 5E-5F show Western blot analysis of mutant HTT, WT HTT and HA (Cas13d constructs containing HA tag) protein levels in zQ175/+ (FIG. 5E) and WT (FIG. 5F) mice treated with either Cas13d-NT or Cas13d-CAG^{EX}, n=3 mice/group. FIGS. 5G-5K show quantification of mutant HTT (FIG. 5G), WT HTT (FIGS. 5H-51) and HA (FIGS. 5J-5K) protein levels in WT and zQ175/+ mice treated with either Cas13d-NT or Cas13d-CAG^{EX} (n=3 biological replicates per experimental group). One-sided Student's t-test. NS, not significant; *P<0.05. P=0.03 in FIG. 5G. No adjustment was made for multiple comparisons.

FIGS. 6A-6I show that Cas13d-CAG^{EX} partially reverses HD-associated differential gene expression in a full-length knock-in mouse model of HD. Cas13d-CAG^{EX} or Cas13d-NT was injected into the striatum of two-month-old HD zQ175 or WT mice and the striatum was collected at ten months of age. Total RNA was extracted and quantified by RNA-seq. n=4 or 5 mice per experimental group. FIGS. 6A-6B show scatter plots of upregulated (FIG. 6A) and downregulated (FIG. 6B) DEGs in zQ175/+ mice treated with either Cas13d-NT or Cas13d-CAG^{EX} AAV9 showing partial reversal of HD-mediated transcriptome changes in HD mouse striatum by Cas13d-CAG^{EX}. FIG. 6C shows volcano plot for DEGs between Q175+Cas13d-CAG^{EX} versus Q175+Cas13d-NT. Most DEGs correspond to a partial to full reversal of HD-associated DEGs. Fold change is relative to Q175+Cas13d-NT. Significance cutoffs are fold change greater ±20% and FDR-adjusted P<0.05. FIGS. 6D-6E show averaged expression of HD-associated upregulated (FIG. 6D) or downregulated (FIG. 6E) DEGs that were significantly reversed by Cas13d-CAG^{EX} treatment. The red dashed line at 0 indicates full rescue to WT levels. ****P<0.0001, two-sample paired Wilcoxon signed-ranks test (n=4 mice per experimental group). The box extends from the first quartile to the third quartile of the data with a line at the median. The whiskers extend from the box by 1.5× the interquartile range. Flier points are those past the end of the whiskers. FIG. 6F shows a scatter plot of upregulated and downregulated DEGs in WT mice treated with either Cas13d-NT or Cas13d-CAG^{EX} AAV9 showing limited off targets. FIGS. 6G-6I show quantification of CNS-specific markers of immunogenicity via qPCR (NeuN (FIG. 6G), GFAP (FIG. 6H), and IBA1 (FIG. 6I)) (one-way ANOVA, Tukey post hoc test, n=3 per experimental group; n=1). Data are presented as mean values±s.e.m.

FIGS. 7A-7F show Cas13d distribution in iPSC-derived neurons with striatal characteristics. FIGS. 7A-7B show quantification of DARPP-32-, and CTIP2-positive cells within day 32 control iPSC-derived neurons with striatal characteristics (3 differentiations per cell lines, 1500 cells per experimental group). FIGS. 7C-7F show image and quantification of Cas13d-HA distribution in Control and HD Day 32 neuronal cultures (n=3 differentiations cell line per experimental group) Data are presented as mean values±SEM. Scale bar=40 μm.

FIGS. 8A-8D show allele-specificity and safety of Cas13d/CAG^{EX} in human iPSC-derived neurons with striatal characteristics. FIG. 8A shows GO analysis of 988 differentially expressed genes that distinguish HD MSN lines from controls as well as HD DEGs reversed by Cas13d/CAGEX. Significant GO terms were determined by Fisher's exact test after FDR correction at p<0.05 and sorted by fold enrichment. FIGS. 8B-8C show quantification of total HTT transcript by TPM and quantitative PCR within control neuronal cultures with Cas13d/NT or Cas13d/CAG^{EX} (n=3 per experimental group) Data are presented as mean values±SEM. FIG. 8D shows a scatter plot of CAG-expanded transcripts in the human transcriptome within control neuronal lines treated with either Cas13d/NT or Cas13d/CAG^{EX} (FDRadjusted p value<0.01).

FIGS. 9A-9F show allele-specificity and safety of Cas13d/CAG^{EX} in a full-length mHTT knock-in mouse model. FIGS. 9A-9B show detection of by Cas13d-HA via HA immunostaining 3 weeks post-intrastriatal injection of AAV9-Cas13d/CAG^{EX}. Scale bar=900 μm (FIG. 9B). FIGS. 9C-9D show Western blot analysis of wild type HTT (wtHTT, antibody MAB 2166 antibody). There are no significant differences between two groups by a one-sided Student's t-test (n=4 mice/group, 2 female and 2 male per group, 11 weeks of age). FIGS. 9E-9F show full images of western blots shown in FIGS. 5E-5F.

FIGS. 10A-10H show body weight, motor function, and the correlation with striatal volume in a full-length mHTT knock-in mouse model of HD. FIGS. 10A-10C show body weight in zQ175/+HD mice or WT mice with indicated treatments. ***p<0.001, two-way ANOVA with Bonferroni post hoc tests for FIG. 4B; ***p<0.001, Mixed-effects model with Bonferroni post hoc tests for FIGS. 4A, 4C. n=10, 5 male and 5 female/group. Data are presented as mean values±SEM. FIGS. 10D-10F show mice were tested on a 5 mm balance beam and time crossing the beam (Traverse time) was recorded at indicated ages in zQ175 HD mice or WT mice with indicated treatments. ***p<0.001, two-way ANOVA with Bonferroni post hoc tests. n=10, 5 male and 5 female/group Data are presented as mean values±SEM.

FIGS. 10G-10H show the correlation analysis (Goodness of Fitness Test) between striatal volume and motor function (FIG. 10G) or body weight (FIG. 10H) in the zQ175/+ mice treated with Cas13d/CAG NT or CAG^{EX}. No significant correlation was detected between striatal volume and motor function (p=0.1236) or body weight (p=0.8455) in the zQ175 treated with either Cas13d/CAG NT or CAG^{EX}.

FIGS. 11A-11O show Cas13d/CAG^{EX} leads to partial reversal of striatum-specific HD disease markers in Q175/+ mice. The results show quantification of striatum-specific HD disease markers via quantitative PCR, the markers including Drd2 (FIG. 11A); Adcy5 (FIG. 11B); Adora2a (FIG. 11C); Ppp1r1b (FIG. 11D); Pde10a (FIG. 11E); Penk (FIG. 11F); Ace (FIG. 11G); Rasd2 (FIG. 11H); Mchr1 (FIG. 11I); Drd1 (FIG. 11J); Dock4 (FIG. 11K); Krt9 (FIG. 11L); Gpr83 (FIG. 11M); Rgs2 (FIG. 11N); and Pdyn (FIG. 11O). CAG^{EX} (one-way ANOVA, Tukey Posthoc Test, **p=0.046, (FIG. 11B) ***p=0.0003, (FIG. 11E) ***p=0.0002, ****p<0.0001; n=3 per experimental group).

FIGS. 12A-12B show Cas13d/CAG^{EX} partially reverses HD-mediated biomarkers in Q175/+ mice. FIG. 12A show GO analysis of upregulated and downregulated DEGs in zQ175/+ as well as HD DEGs reversed by Cas13d/CAG^{EX}. Significant GO terms were determined by Fisher's exact test after FDR correction at p<0.05 and sorted by fold enrichment. FIG. 12B show overlay of reported Q175/+DEGs in Morelli et al and those previously reported in Landfelder et al and Obenauer et al with a significance threshold of FDR-adjusted p-value<0.01.

FIGS. 13A-13B show Cas13d/CAG^{EX} causes limited off-target effects on the mouse transcriptome in vivo. FIG. 13A shows a scatter plot of CAG-expanded transcripts in the mouse transcriptome within Q175/+ mice treated with either Cas13d/NT or Cas13d/CAG^{EX} (FDR-adjusted p-value<0.01). FIG. 13B shows a scatter plot of CAG-expanded transcripts in the mouse transcriptome within WT mice treated with either Cas13d/NT or Cas13d/CAG^{EX} (FDR-adjusted p-value<0.01).

### DETAILED DESCRIPTION

The present disclosure describes a method of treating Huntington's disease in a subject that includes administering to the subject a therapeutically effective amount of a pharmaceutical composition comprising: (a) a guide RNA (gRNA), wherein the guide RNA comprises a repeat sequence complementary to a target CAG-expansion RNA; and (b) a CRISPR-associated protein or a nucleic acid sequence encoding the CRISPR-associated protein, wherein the CRISPR-associated protein comprises a Cas13d polypeptide, wherein the gRNA allele-selectively targets the target CAG-expansion RNA. Also described herein are recombinant allele-selective expression systems for CRISPR/Cas-directed RNA targeting of a target CAG-expansion RNA comprising: (a) a guide RNA, wherein the guide RNA comprises a repeat sequence complementary to the target CAG-expansion RNA; and (b) a CRISPR-associated protein or a nucleic acid sequence encoding the CRISPR-associated protein, wherein the CRISPR-associated protein comprises a Cas13d polypeptide.

Various non-limiting aspects of these methods are described herein, and can be used in any combination without limitation. Additional aspects of various components of methods for regulating gene expression are known in the art.

It must be noted that, as used in the specification and the appended claims, the singular forms “a,” “an” and “the” include plural referents unless the context clearly dictates otherwise.

As used herein, the term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, a “cell” can refer to either a prokaryotic or eukaryotic cell, optionally obtained from a subject or a commercially available source.

As used herein, “delivering”, “gene delivery”, “gene transfer”, “transducing” can refer to the introduction of an exogenous polynucleotide into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery molecule. Examples of gene delivery molecules can include, but are not limited to, liposomes, micelle biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein, the term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “exogenous” refers to any material introduced from or originating from outside a cell, a tissue or an organism that is not produced by or does not originate from the same cell, tissue, or organism in which it is being introduced.

As used herein, the term “expression” refers to the process by which polynucleotides are transcribed into mRNA and/or the process by which the transcribed mRNA is subsequently translated into peptides, polypeptides, or proteins. In some embodiments, if the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, “nucleic acid” is used to include any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

In some embodiments, the term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

As used herein, the term “nucleotides” and “nt” are used interchangeably herein to generally refer to biological molecules that comprise nucleic acids. Nucleotides can have moieties that contain the known purine and pyrimidine bases. Nucleotides may have other heterocyclic bases that have been modified. Such modifications include, e.g., methylated purines or pyrimidines, acylated purines or pyrimidines, alkylated riboses, or other heterocycles. In some embodiments, nucleic acid modifications can also include a blocking modification comprising a 3′ end modification (e.g., a 3′ dideoxy C (3′ddC), 3′ddG, 3′ddA, 3′ddT, 3′ inverted dT, 3′ C3 spacer, 3′ amino, 3′ biotinylation, or 3′ phosphorylation). The terms “polynucleotides,” “nucleic acid,” and “oligonucleotides” can be used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise non-naturally occurring sequences. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, the term “plurality” can refer to a state of having a plural (e.g., more than one) number of different types of things (e.g., a cell, a genomic sequence, a subject, a system, or a protein). In some embodiments, a plurality of genomic sequences can be more than one genomic sequence wherein each genomic sequence is different from each other.

As used herein, the term “recombinant” refers to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

### A. Huntington's Disease (HD)

Huntington's disease (HD) is an autosomal dominant neurodegenerative disorder caused by a CAG short tandem repeat (STR) expansion in exon 1 of the huntingtin (HTT) gene 1. This trinucleotide sequence codes for the amino acid glutamine (Q), placing HD in a broader class of neurological disorders known as polyglutamine (polyQ) diseases. Motor symptoms can manifest from childhood to old age, with onset inversely correlated with CAG repeat length in mutant HTT, with the longer the CAG repeat, the earlier symptoms arise. In some embodiments, juvenile HD patients can present longer CAG repeat lengths in mutant HTT. In some embodiments, a juvenile HD patient is under the age of 18 years old (e.g., 17 years old, 16 years old, 15 years old, 14 years old, 13 years old, 12 years old, 11 years old, 10 years old, 9 years old, 8 years old, 7 years old, 6 years old, 5 years old, 4 years old, 3 years old, or 2 years old). In some embodiments, a juvenile-onset HD patient can present a CAG repeat length of more than 70 (e.g., more than 100, more than 200, or more than 300) copies of the CAG repeat sequence. In some embodiments, juveniles may have between about 30 and 60 copies of the CAG repeat. Juveniles may have more than 30 copies of the CAG repeat, but not have clinical symptoms of HD. Juveniles may have more than 30 copies of the CAG repeat, but have pre-clinical symptoms. In some embodiments, adult-onset HD patients (e.g., over the age of 18 years old) can present between about 35 to about 65 (e.g., about 40 to about 65, about 45 to about 65, about 50 to about 65, about 55 to about 65, about 60 to about 65, about 35 to about 60, about 40 to about 60, about 45 to about 60, about 50 to about 60, about 55 to about 60, about 35 to about 55, about 40 to about 55, about 45 to about 55, about 50 to about 55, about 35 to about 50, about 40 to about 50, about 45 to about 50, about 35 to about 45, about 40 to about 45, or about 35 to about 40) copies of the CAG repeat sequence in a mutant HTT. In some embodiments, adult-onset HD patients (e.g., over the age of 18 years old) can present between about 30 to about 60 (e.g., about 35 to about 60, about 40 to about 60, about 45 to about 60, about 50 to about 60, about 55 to about 60, about 30 to about 55, about 35 to about 55, about 40 to about 55, about 45 to about 55, about 50 to about 55, about 30 to about 50, about 35 to about 50, about 40 to about 50, about 45 to about 50, about 30 to about 45, about 35 to about 45, about 40 to about 45, about 30 to about 40, about 35 to about 40, or about 30 to about 35) copies of the CAG repeat sequence in a mutant HTT. In some embodiments, adults with HD may have more than 60 copies of the CAG repeat. Adults with HD may have more than 30 copies of the CAG repeat, but not have clinical symptoms of HD. Adults may have more than 30 copies of the CAG repeat, but have pre-clinical symptoms.

The therapies currently available to patients with HD offer only moderate symptomatic relief and affected individuals typically die 15-20 years post-diagnosis due to complications. Therefore, a major focus of HD therapeutic development has shifted toward targeting the root of the disease through depletion of mutant HTT. In some embodiments, besides the toxicity of the mutated HTT protein, mutant HTT mRNA can also contribute to disease pathogenesis. Consequently, strategies to suppress both HTT transcripts and protein levels would be most beneficial as a treatment.

RNA interference (RNAi) and antisense oligonucleotide (ASO) strategies have shown preclinical efficacy. However, most of these approaches do not precisely differentiate mutant HTT from the normal allele. Most patients with HD are heterozygous for the CAG expansion and rely on their normal HTT allele to play important roles during brain development as well as in adult central nervous system (CNS) function. In the adult brain, HTT helps regulate intracellular vesicle trafficking, transcriptional regulation and synaptic connectivity. In some embodiments, sustained reduction of normal HTT levels may even exacerbate HD pathogenesis. In some embodiments, an allele-selective system can selectively and effectively suppress mutant HTT mRNA expression while sparing the normal (wildtype) allele of HTT, thereby providing a method of treating HD.

### B. Recombinant Allele-Selective Expression System

Provided herein are recombinant allele-selective expression systems for CRISPR/Cas-directed RNA targeting of a target CAG-expansion RNA comprising: (a) a guide RNA, wherein the guide RNA comprises a repeat sequence complementary to the target CAG-expansion RNA; and (b) a CRISPR-associated protein or a nucleic acid sequence encoding the CRISPR-associated protein, wherein the CRISPR-associated protein comprises a Cas13d polypeptide

As used herein, “allele-selective” can refer to the discrimination of a mutant allele from a wild-type allele. In some embodiments, an allele-selective system identifies a mutant allele from a wild-type allele by recognizing a specific nucleic acid (e.g., DNA, RNA) sequence of the mutant allele. In some embodiments, an allele-selective system identifies a mutant allele from a wild-type allele by recognizing a specific mutation within the HTT transcript. In some embodiments, an allele-selective system identifies a mutant allele from a wild-type allele by recognizing single nucleotide polymorphisms (SNPs) within the HTT transcript. In some embodiments, an allele-selective system identifies a mutant allele from a wild-type allele by recognizing a specific secondary structure of a RNA molecule of the mutant allele. For example, in some embodiments, an allele-selective system identifies the mutant allele of the HTT allele, wherein the mutant HTT comprises a CAG-expansion sequence. In some embodiments, the allele-selective system recognizes the CAG-expansion sequence itself, wherein a mutant HTT comprises a longer CAG-expansion sequence (e.g., more than 30 copies, more than 60 copies, or more than 100 copies). In some embodiments, the allele-selective system recognizes associated SNPs that are found preferentially in the mutant HTT transcript. In some embodiments, the allele-selective system recognizes a double-stranded RNA structure such a self-annealing hairpin structure of the mutant HTT. In some embodiments, the allele-selective system recognizes a secondary structure of the RNA molecule (e.g., hairpin structure) of the mutant HTT.

As used herein, “repeat expansion sequence” or “repeat sequence” can refer to short or long patterns of nucleic acids (e.g., DNA or RNA) that occur in multiple copies throughout the genome. In some embodiments, these repeated sequences are necessary for maintaining important genome structures such as telomeres or centromeres. In some embodiments, repeated sequences can be important for cellular functioning and genome maintenance, while other repetitive sequences can be harmful. For example, many repetitive RNA sequences have been linked to human diseases such as Huntington's disease and Friedreich's ataxia. In some embodiments, Huntington's disease can be linked to an expansion of CAG repeat sequences in the huntingtin (HTT) gene. In some embodiments, a subject diagnosed with HD can have a normal HTT allele and a mutant HTT allele, wherein the mutant allele comprises a CAG-expansion RNA.

In some embodiments, a subject can have a normal HTT allele, wherein the normal HTT allele comprises less than 30 copies of CAG repeat sequences. In some embodiments, a subject that has been diagnosed as having Huntington's disease can have a mutant HTT allele, wherein the mutant HTT allele comprises a CAG-expansion RNA. In some embodiments, the target CAG-expansion RNA comprises more than 30 copies of a target CAG-expansion RNA sequence. In some embodiments, the target CAG-expansion RNA comprises between 31 and 60 copies of a CAG repeat sequence. In some embodiments, the target CAG-expansion RNA comprises more than 60 copies of a CAG repeat sequence. In some embodiments, the target CAG-expansion RNA comprises more than 100 copies of a CAG repeat sequence. In some embodiments, the target CAG-expansion RNA comprises a mutant huntingtin gene (HTT).

### CRISPR/Cas Systems

In some embodiments, CRISPR/Cas-directed RNA targeting can include CRISPR components. For example, in some embodiments, CRISPR components can include, but are not limited to, a guide RNA (gRNA) and a CRISPR-associated endonuclease (Cas protein). In some embodiments, CRISPR/Cas-directed RNA targeting comprises (a) a guide RNA, wherein the guide RNA comprises a repeat sequence complementary to a target CAG-expansion RNA; and (b) a CRISPR-associated protein or a nucleic acid sequence encoding the CRISPR-associated protein, wherein the CRISPR-associated protein comprises a Cas 13d polypeptide.

As used herein, the term “CRISPR” refers to a technique of sequence specific genetic manipulation relying on the clustered regularly interspaced short palindromic repeats pathway, which unlike RNA interference regulates gene expression at a transcriptional level. As used herein, a “Cas effector” or “CRISPR-associated protein” can refer to an enzyme or protein that uses CRISPR sequences as a guide to recognize and cleave specific nucleic acid strands that are complementary to the CRISPR sequence. In some embodiments, a CRISPR-associated protein can comprise a Cas13d polypeptide. In some embodiments, the CRISPR-associated protein can comprise a Cas9 protein, a Cas13b protein, or a Cas13d protein. In some embodiments, the CRISPR-associated protein can comprises a Cas13a protein, a Cas13b protein, a Cas 13d proteins, or a Cas 13g protein. In some embodiments, the CRISPR-associated protein can include a Cas9 endonuclease that makes a double-stranded break in a target DNA sequence. In some embodiments, the CRISPR-associated protein can be a Cas12a nuclease that also makes a double-stranded break in a target DNA sequence. In some embodiments, the CRISPR-associated protein can be a Cas13 nuclease which targets RNA. In some embodiments, the CRISPR-associated protein comprises a nuclease dead Cas9 (dCas9) protein. In some embodiments, the CRISPR-associated protein comprises a Cas13b protein.

In some embodiments, the CRISPR-associated protein comprises a Cas13d protein. Cas13d is a compact RNA-targeting type VI CRISPR-associated protein, with a size of approximately 930 amino acids. Cas13d have dual RNase activities and is capable of cleaving target RNA with no target-flanking sequence requirements. In some embodiments, the Cas13d protein is Ruminococcus Flavefaciens XPD3002 (Rfx) Cas13d. In some embodiments, the Cas13d is tagged with an HA epitope. In some embodiments, the nucleic acid sequence encoding the Cas13d polypeptide further comprises an RNA polymerase II promoter sequence or an RNA polymerase III promoter sequence. In some embodiments, the RNA polymerase III promoter sequence is an EF1a promoter sequence.

As used herein, the term “gRNA” or “guide RNA” refers to the guide RNA sequences used to target specific genes for employing the CRISPR technique. Techniques of designing gRNAs and donor therapeutic polynucleotides for target specificity are well known in the art. For example, Doench, J., et al. Nature biotechnology 2014; 32 (12): 1262-7 and Graham, D., et al. Genome Biol. 2015; 16:260.

In some embodiments, the guide RNA can recognize a target RNA, for example, by hybridizing to the target RNA. In some embodiments, the guide RNA comprises a sequence that is complementary to the target RNA. In some embodiments, the gRNA can include one or more modified nucleotides. In some embodiments, the gRNA has a length that is about 10 nt (e.g., about 20 nt, about 30 nt, about 40 nt, about 50 nt, about 60 nt, about 70 nt, about 80 nt, about 90 nt, about 100 nt, about 120 nt, about 140 nt, about 160 nt, about 180 nt, about 200 nt, about 300 nt, about 400 nt, about 500 nt, about 600 nt, about 700 nt, about 800 nt, about 900 nt, about 1000 nt, or about 2000 nt).

In some embodiments, a guide RNA can recognize a target CAG-expansion RNA, wherein the gRNA comprises a repeat sequence complementary to the target CAG-expansion RNA. In some embodiments, the gRNA is an allele-specific or allele-targeting gRNA, wherein the gRNA selectively binds to mHTT but not normal (wildtype) HTT transcripts. In some embodiments, the repeat sequence of the guide RNA comprises GTC, TCG, or CGT. In some embodiments, the guide RNA comprises a sequence selected from the group consisting of SEQ ID NO: 1, SEQ ID NO: 2, or SEQ ID NO: 3 (Table 1).

TABLE 1

| gRNA Name | gRNA Sequence | SEQ ID NO |
|---|---|---|
| CAG^{EX}-1 gRNA | GTCGTCGTCGTCGTCGTCGTC | 1 |
| CAG^{EX}-2 gRNA | TCGTCGTCGTCGTCGTCGTCG | 2 |
| CAG^{EX}-3 gRNA | CGTCGTCGTCGTCGTCGTCGT | 3 |

In some embodiments, a guide RNA can recognize a variety of RNA targets. For example, a target RNA can be messenger RNA (mRNA), ribosomal RNA (rRNA), signal recognition particle RNA (SRP RNA), transfer RNA (tRNA), small nuclear RNA (snRNA), small nucleolar RNA (snoRNA), antisense RNA (aRNA), long noncoding RNA (lncRNA), microRNA (miRNA), piwi-interacting RNA (piRNA), small interfering RNA (siRNA), short hairpin RNA (shRNA), retrotransposon RNA, viral genome RNA, or viral noncoding RNA. In some embodiments, a target RNA can be an RNA involved in pathogenesis of conditions such as repeat expansion diseases. In some embodiments, a target RNA can be a therapeutic target for conditions such as Huntington's disease. In some embodiments, the gRNA further comprises a promoter sequence. In some embodiments, the gRNA can be driven by a promoter. In some embodiments, the promoter can be a U6 polymerase III promoter. In some embodiments, the gRNA further comprises a U6 promoter sequence.

### C. Vectors

Also provided herein are vectors comprising a nucleic acid encoding (i) a guide RNA, wherein the guide RNA comprises a repeat sequence complementary to a target CAG-expansion RNA, and (ii) a CRISPR-associated protein, wherein the CRISPR-associated protein comprises a Cas13d polypeptide, wherein the gRNA allele-selectively targets the target CAG-expansion RNA.

In some embodiments, a vector comprises at least one of (a) the gRNA and (b) the CRISPR-associated protein. In some embodiments, the vector comprises both (a) and (b).

In some embodiments, the vector is a viral vector. In some embodiments, the viral vector includes a sequence isolated or derived from a retrovirus. In some embodiments, the viral vector includes a sequence isolated or derived from a lentivirus. In some embodiments, the viral vector includes a sequence isolated or derived from an adenovirus. In some embodiments, the viral vector includes a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments, the viral vector includes a sequence isolated or derived from an adeno-associated virus (AAV). In some embodiments, the viral vector includes an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11, AAV12, AAV.rh32/33, AAV.rh43, AAV.rh64R1, and any combinations or equivalents thereof. In some embodiments, the viral vector is replication incompetent. In some embodiments, the viral vector is isolated or recombinant (rAAV). In some embodiments, the viral vector is self-complementary (scAAV). In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 4.5 kb to 4.75 kb.

In some embodiments, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

In some embodiments, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. As used herein, a “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting exemplary promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nß2, PPE, ENK, EAAT2, GFAP, MBP, and U6 promoters. An “enhancer” is a region of DNA that can be bound by activating proteins to increase the likelihood or frequency of transcription. Non-limiting exemplary enhancers and posttranscriptional regulatory elements include the CMV enhancer and WPRE.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art (see, e.g., Trono D. (2002) Lentiviral vectors, New York: Spring-Verlag Berlin Heidelberg and Durand et al. (2011) Viruses 3 (2): 132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIVsM) vector, a modified sooty mangabey simian immunodeficiency virus (SIVsM) vector, a African green monkey simian immunodeficiency virus (SIVAGm) vector, a modified African green monkey simian immunodeficiency virus (SIVAGm) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

In some embodiments, the vector can be introduced into any cell, e.g., a mammalian cell. Non-limiting examples of a mammalian cell include: a human cell, a rodent cell (e.g., a rat cell or a mouse cell), a rabbit cell, a dog cell, a cat cell, a porcine cell, or a non-human primate cell. In some embodiments, the vector can be delivered into the cytoplasm of a cell. In some embodiments, the vector can be delivered into the cell by chemical transfection, non-chemical transfection, particle-based transfection, or viral transfection. In some embodiments, the vector can be delivered with a transfection reagent.

### D. Methods of Treating Huntington's Disease

Provided herein are methods of treating Huntington's disease in a subject, the method comprising administering to the subject a therapeutically effective amount of a pharmaceutical composition comprising: (a) a guide RNA (gRNA), wherein the guide RNA comprises a repeat sequence complementary to a target CAG-expansion RNA, and wherein the gRNA allele-selectively targets the target CAG-expansion RNA; and (b) a CRISPR-associated protein or a nucleic acid sequence encoding the CRISPR-associated protein, wherein the CRISPR-associated protein comprises a Cas13d polypeptide.

In some embodiments, the methods can include the administration of pharmaceutical compositions and formulations including vectors delivering a recombinant allele-selective expression system that includes a gRNA and a CRISPR-associated protein.

### Pharmaceutical Compositions

In some embodiments, the pharmaceutical compositions are formulated with a pharmaceutically acceptable carrier. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The pharmaceutical composition can be administered alone or as a component of a pharmaceutical formulation. The compounds may be formulated for administration, in any convenient way for use in human or veterinary medicine. The compositions may conveniently be presented in unit dosage form and may be prepared by any methods well known in the art of pharmacy. The amount of active ingredient which can be combined with a carrier material to produce a single dosage form can vary depending upon the host being treated, the particular mode of administration. The amount of active ingredient which can be combined with a carrier material to produce a single dosage form will generally be that amount of the compound which produces a therapeutic effect.

Pharmaceutical compositions described herein can be prepared according to any method known to the art for the manufacture of pharmaceuticals. Such compositions can contain, for example, preserving agents. A composition can be admixtured with nontoxic pharmaceutically acceptable excipients which are suitable for manufacture. Compositions may comprise one or more diluents, emulsifiers, preservatives, buffers, excipients, etc. and may be provided in such forms as liquids, powders, emulsions, lyophilized powders, controlled release formulations, on patches, in implants, etc. Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences as described herein. As an example of an injectable oil vehicle, see Minto (1997) J. Pharmacol. Exp. Ther. 281:93-102.

Pharmaceutical compositions can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as egg or soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

In some embodiments, the pharmaceutical compositions can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compositions can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., US20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

### Administration

In some embodiments, the administration of the pharmaceutical composition comprises intrastriatal administration. In some embodiments, the administration of the pharmaceutical composition comprises ocular, oral, parenteral, bronchial (e.g., by bronchial instillation), buccal, enteral, intra-arterial, intragastric, intramedullary, intramuscular, intranasal, intraperitoneal, intrathecal, intravenous, intraventricular, intracisternal, within a specific organ (e.g., intrahepatic), mucosal, nasal, oral, rectal, transcutaneous, subcutaneous, sublingual, tracheal (e.g., by intratracheal instillation), vaginal, or vitreal. In some embodiments, the pharmaceutical composition is administered by enteral administration, intraperitoneal administration, intravenous administration, intramuscular administration, subcutaneous administration, intracutaneous administration, oral administration, intranasal administration, intrapulmonary administration, intrarectal administration, intrastriatal administration or a telemetry controlled external or implanted infusion pump.

In some embodiments, the subject is diagnosed as having Huntington's disease. In some embodiments, the subject is under 18 years old. In some embodiments, the subject is older than 18 years old.

In some embodiments, the administration of the pharmaceutical composition reduces mRNA and/or protein expression of the target CAG-expansion RNA.

### EXAMPLES

### Example 1—Development of a Cas13d System that Targets CAG^{EX}

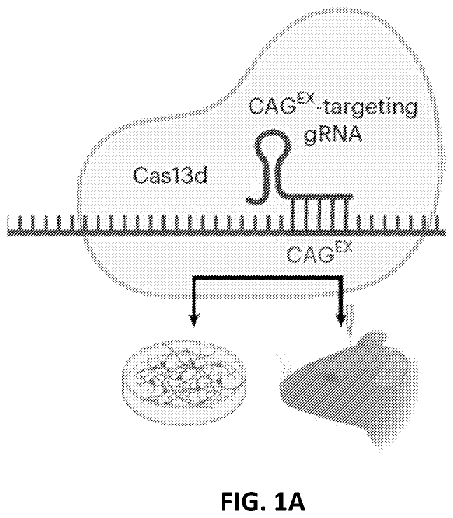

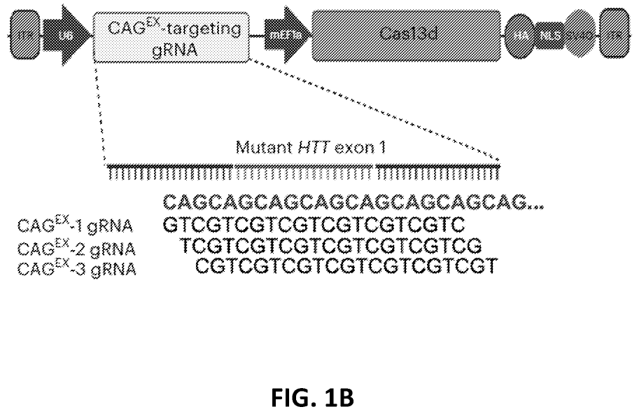

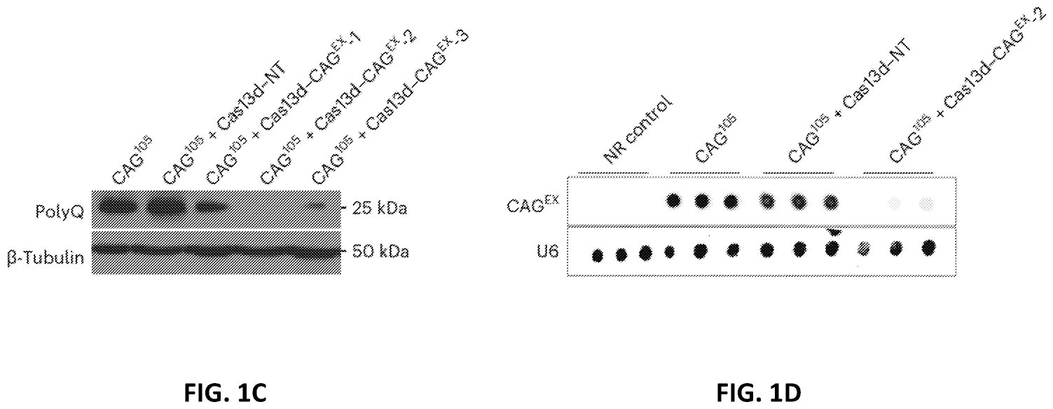

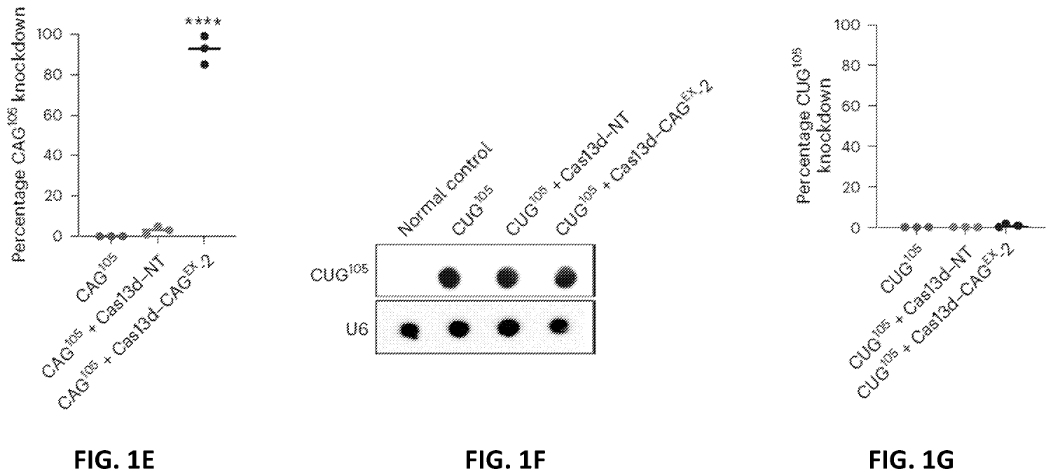

Although the etiology of HD is complex, many proposed mechanisms arise from the transcription and subsequent translation of CAG^{EX} HTT, ultimately causing disease through a toxic gain-of-function mechanism. The methods described herein used a Cas9 or Cas13d system to target and eliminate toxic repeat RNAs in vitro and in vivo delivered by a two-vector AAV system, demonstrating that RNA-targeting CRISPR approaches are effective with RNA repeat expansions. Here, due to the smaller size and natural RNA-targeting capacity of Ruminococcus flavefaciens XPD3002 (Rfx) CRISPR-Cas13d, a CAG^{EX} RNA-targeting Cas13d (Cas13d-CAG^{EX}) system was developed that was packaged into a single vector in both lentiviral and AAV delivery vehicles to evaluate its therapeutic potential in multiple established preclinical models of HD. These included fibroblasts from patients with HD, differentiated neurons with striatal characteristics from a panel of induced pluripotent stem cell (iPSC) lines from patients with HD and a full-length mutant HTT knock-in mouse model expressing a human mutant exon-1 with the expanded CAG repeat (approximately 220 repeats) within the native mouse huntingtin gene, zQ175/+ (FIG. 1A).

First, to optimize knockdown of CAG^{EX} HTT RNA by Cas13d, three distinct RNA-targeting vectors were engineered that consist of (1) Cas13d tagged with a human influenza hemagglutinin (HA) epitope and (2) one of three U6 promoter-driven Cas13d gRNAs (denoted as CAG^{EX} gRNA 1-3). All three gRNAs are complementary to the CAG^{EX} RNA sequence with each guide targeting a different codon within the repeat expansion: CAG^{EX} gRNA-1 (GTC), CAG^{EX} gRNA-2 (TCG) and CAG^{EX} gRNA-3 (CGT) (FIG. 1B). For an initial assessment of the capability of each CAG^{EX} gRNA to knockdown CAG^{EX} RNA, HEK293 cells were cotransfected with a repeat expansion plasmid with 105 CAG STRs along with a Cas13d-containing vector with a nontargeting gRNA designed to target a sequence from the λ bacteriophage (Cas13d-NT) or one of the three CAG^{EX}-targeting guides. Since aggregation of toxic polyQ protein translated from CAG^{EX} RNA is well documented as one of the primary hallmarks of HD neuropathology, it was determined if and to what extent Cas13d in conjunction with each CAG^{EX}-targeting gRNA can eliminate polyQ protein in live human cells. It was observed that polyQ protein produced by this plasmid was dramatically reduced in cells cotransfected with Cas13d and CAG^{EX} gRNA-2 compared to cells only transfected with the CAG105-expressing plasmid via western blot with a polyQ-specific antibody (MAB1574) (FIG. 1C). RNA blots performed with RNA isolated from cells cotransfected with the CAG repeat expression plasmid and Cas13d with the CAG^{EX}-2 gRNA also showed a significant (85.4%) reduction of CAG^{EX} RNA compared with cells transfected with the nontargeting Cas13d vector (one-way analysis of variance (ANOVA), ****P<0.001) (FIG. 1D, quantified in FIG. 1E). Importantly, expression studies using CUG expansion (CUG^{EX}) RNA-expressing plasmids and a semiquantitative RNA dot blot were performed to determine if Cas13d with the CAG^{EX}-2 gRNA specifically eliminates RNA transcripts that contain CAG expansions without degrading similar GC-rich transcripts such as those with CUG expansions. It was observed that Cas13d with the CAG^{EX}-2 gRNA specifically targets and degrades CAG^{EX} transcripts while leaving CUG^{EX} transcripts intact (FIG. 1F, quantified in FIG. 1G). Taken together, these data confirm that the provided Cas13d system, which includes the CAG^{EX}-2 gRNA, now referred to as ‘Cas13d-CAG^{EX}’, can effectively eliminate CAG^{EX} RNA and subsequent polyQ protein in human cells possibly without targeting other trinucleotide repetitive elements such as CUG^{EX}.

### Example 2—Cas13d-CAG^{EX} Reduces mHTT in Cells from Patients with HD

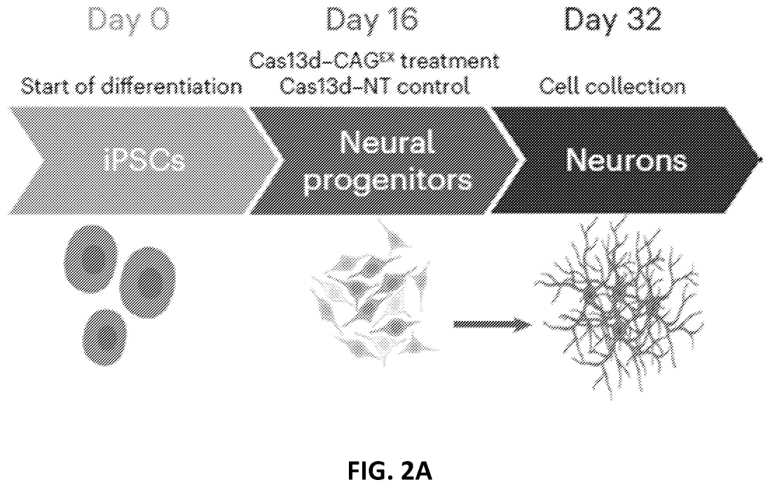

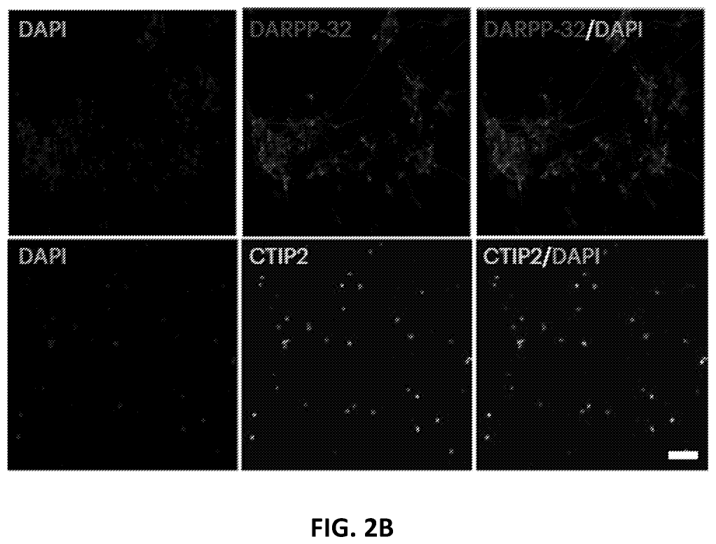

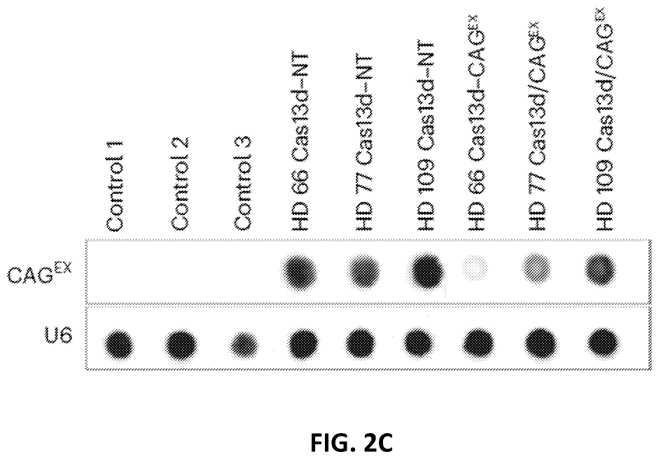

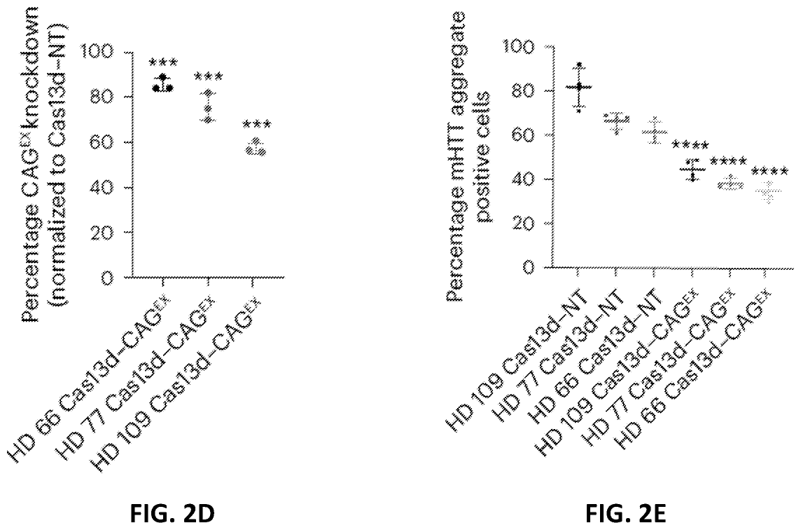

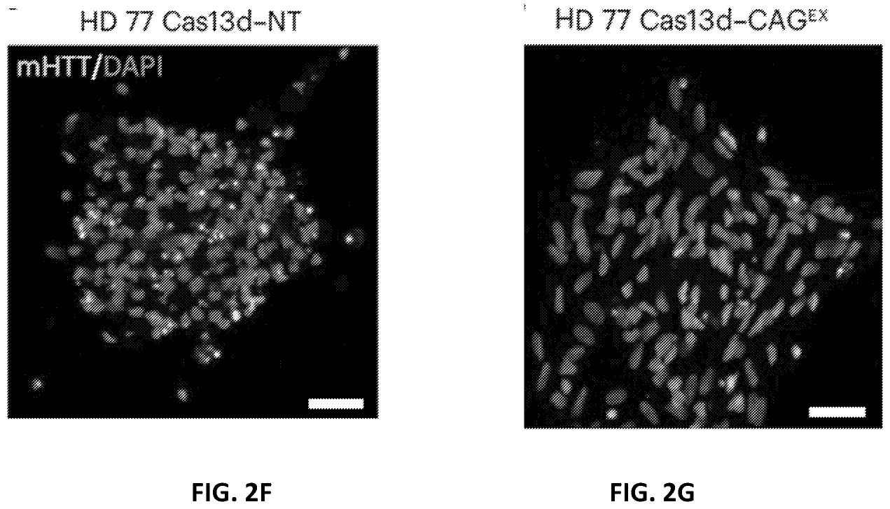

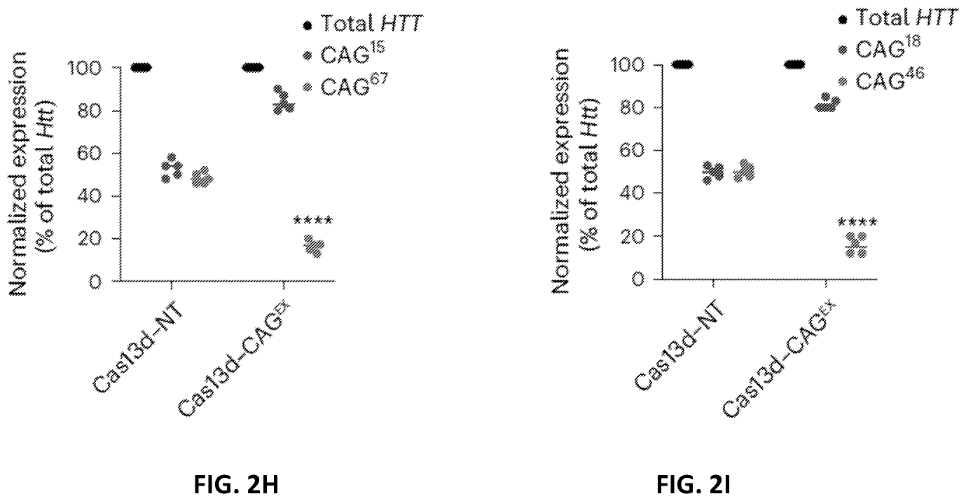

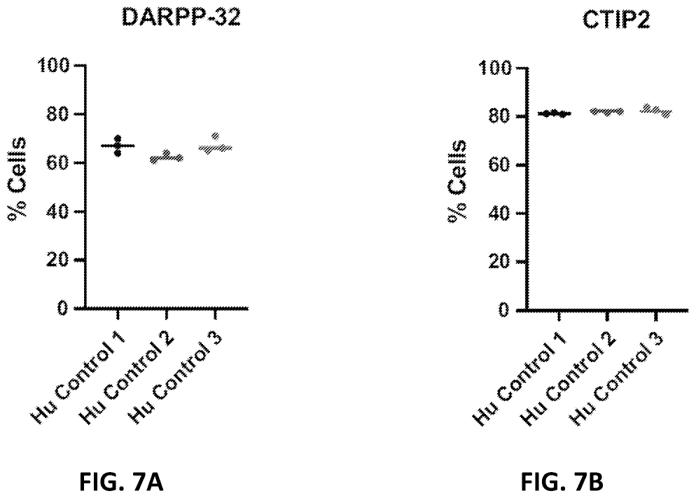

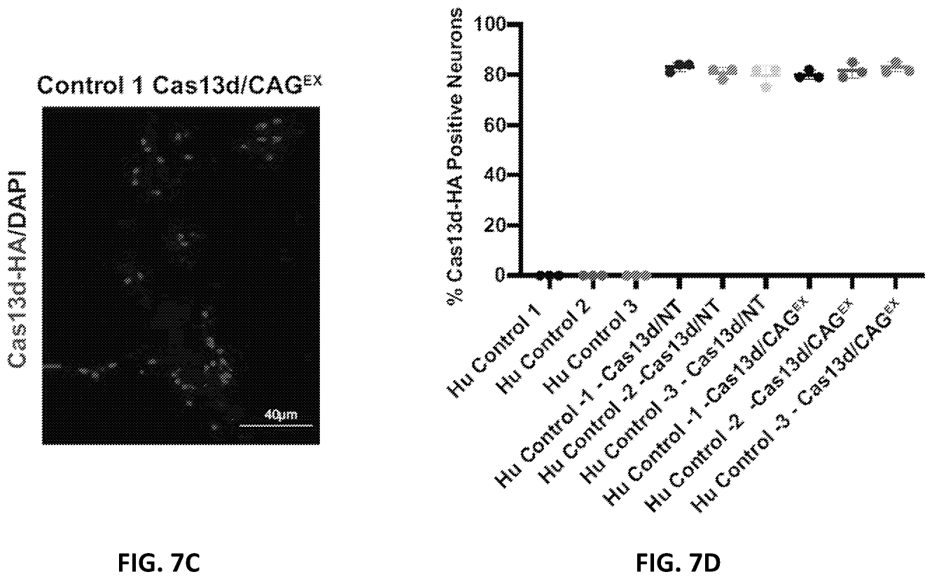

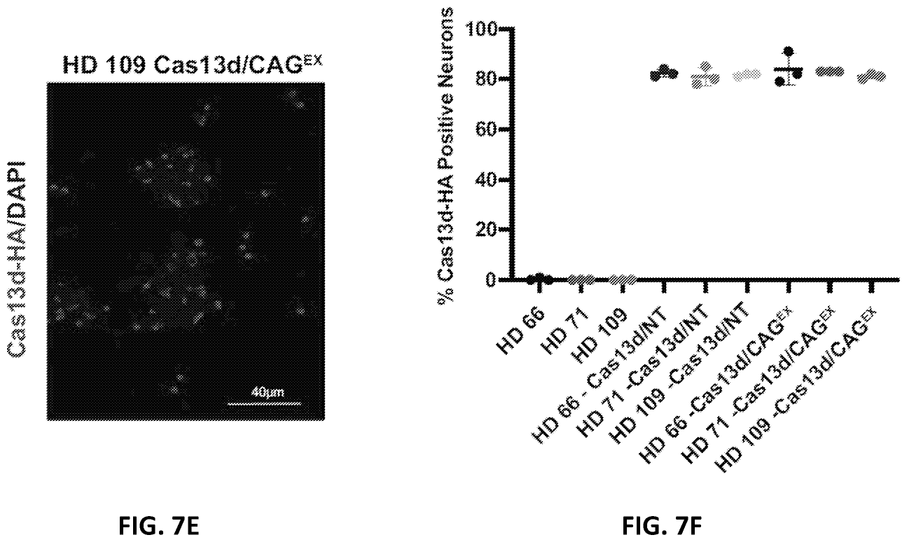

To evaluate Cas13d-CAG^{EX} in a human preclinical model, a panel of neuronal cultures enriched for striatal characteristics was generated from three previously validated iPSC lines derived from individual patients with HD. These independent lines contained repeats in the HTT locus ranging from 66 to 109 CAGs (referred to as HD 66, 77 and 109) and were compared to 3 non-isogenic, neurotypical iPSC lines isolated from three different individuals with <25 CAG repeats in exon 1 of HTT. Although not age- or sex-matched to cell lines from patients with HD, the control samples (controls 1-3) used to evaluate the effects of different lengths of CAG expansions on human neurons have been characterized and checked for aberrant genomic alterations via karyotype and copy number variation (CNV) arrays to avoid line-specific confounds in downstream analyses. Each HD and control neuronal culture was transduced with a constitutive, lentiviral system supplying either Cas13d-CAG^{EX} or nontargeting Cas13d-NT at 16 days post-differentiation, when most of each culture consisted of neuroprogenitor cells (FIG. 2A). At day 32 post-differentiation, both HD and control cultures consisted of neurons positive for striatal markers, dopamine- and cAMP-regulated neuronal phosphoprotein (DARPP-32) and COUP-TF1-interacting protein 2 (CTIP2) (FIG. 2B, quantified in FIGS. 7A-7B). A high percentage of DARPP-32 (65.5%) and CTIP2 (82.3%) was observed indicating that human cell cultures consisted of neurons enriched for striatal characteristics. Widespread transduction was confirmed with Cas13d-HA immunofluorescence with 78% or more (average 83.4%) of neurons showing Cas13d expression (FIGS. 7C-7F). RNA slot blot hybridization detected that CAG^{EX} RNA was significantly reduced by Cas13d-CAG^{EX} with an 84.1% reduction in HD 66, a 79.8% reduction in HD 77 and a 56.2% reduction in HD 109 (FIG. 2C, quantified in FIG. 2D). Since protein aggregates are pathological hallmarks of HD, the effect of Cas13d-CAG^{EX} on mutant HTT aggregation was determined, immunolabeled by the EM48 antibody. Cas13d-CAG^{EX}-treated HD lines had significantly reduced mutant HTT aggregates compared with those treated with Cas13d-NT (FIG. 2E, representative images in FIG. 2F-2G), indicating that Cas13d-CAG^{EX} can selectively suppress mutant HTT aggregates.

To evaluate the allele-selectivity of Cas13d-CAG^{EX} on shorter repeats more common in adult-onset patients, two human fibroblast lines from patients with HD with (67/15) CAG repeats (GM04723) and (46/18) CAG repeats (GM02151) were utilized. Allele-specific quantitative PCR with reverse transcription (RT-qPCR) using patient-specific single-nucleotide polymorphism (SNP)-based primers showed a significant and specific knockdown of mutant HTT RNA in each patient fibroblast line treated with Cas13d-CAG^{EX}-expressing lentiviral vectors compared to those transfected with Cas13d-NT (FIGS. 2H-2I). These data support the broad utility of Cas13d-CAG^{EX} for patients with adult- or juvenile-onset HD-relevant CAG repeat lengths in mutant HTT.

### Example 3—Cas13d-CAG^{EX} Reduces Specific Molecular Biomarkers in Patients with HD

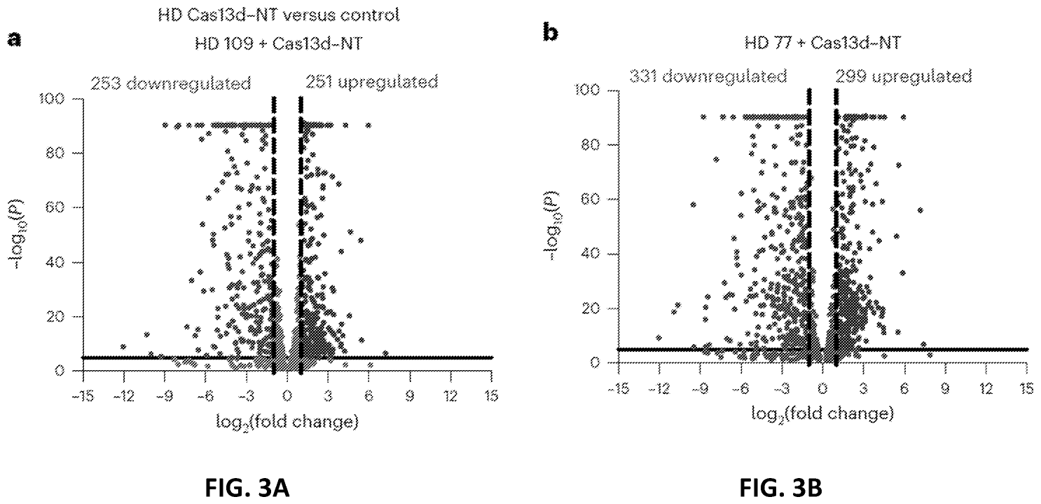

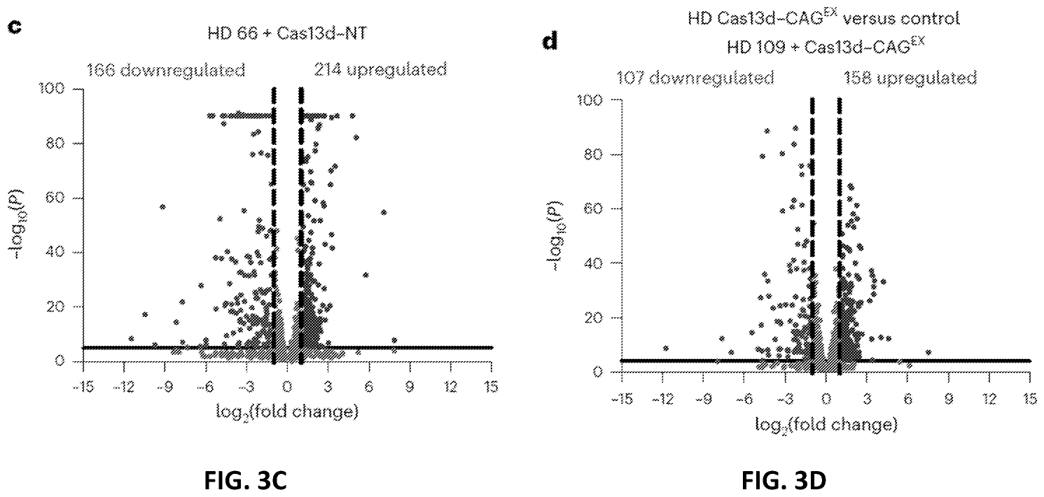

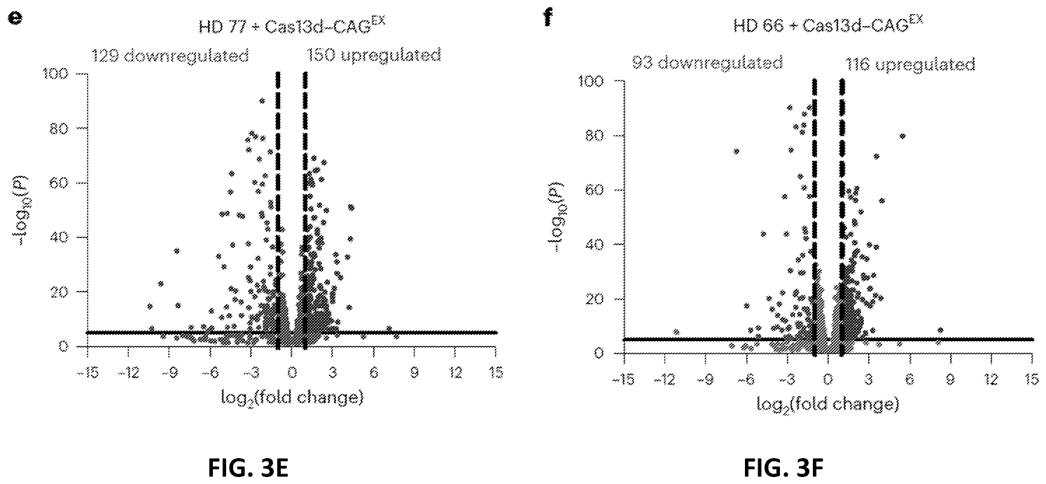

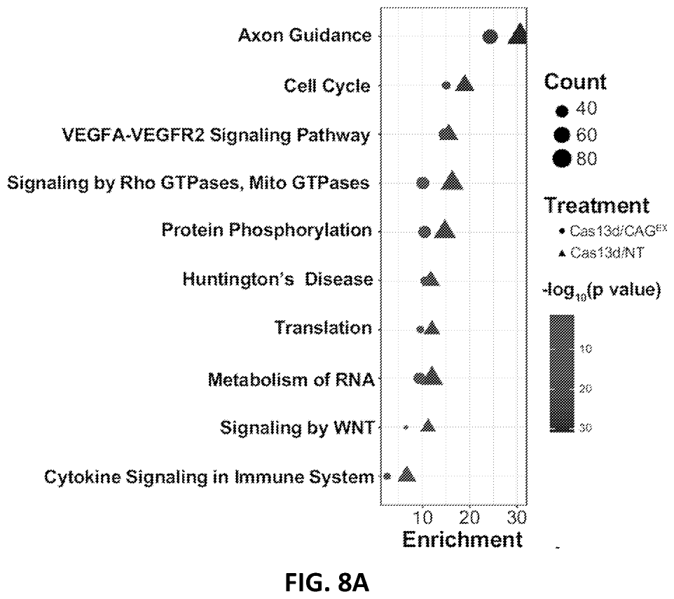

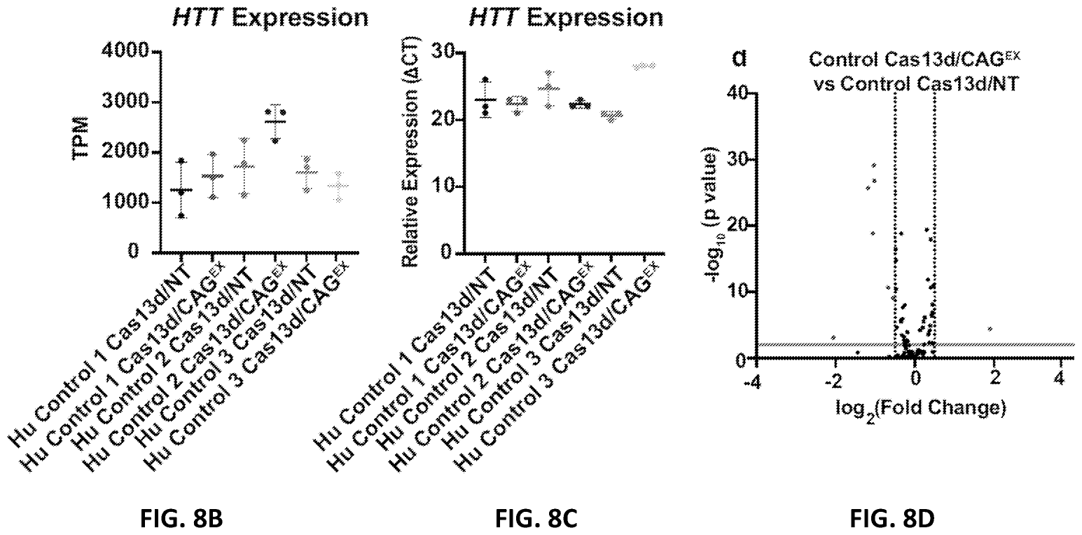

Transcriptome-wide RNA sequencing (RNA-seq) analysis utilizing the DESeq2 package identified 988 differentially expressed genes (DEGs) (false discovery rate (FDR)-adjusted P<0.0001) that distinguish the HD neuronal lines from controls (FIGS. 3A-3C). Gene ontology (GO) biological process analyses revealed that these DEGs were enriched for terms associated with neurodegenerative disorders including ‘Rho GTPases’, ‘translation’ and the ‘Vascular endothelial growth factor A-vascular endothelial growth factor receptor 2 signaling pathway’. The term ‘Huntington disease’ was also enriched and included genes associated with known HD-mediated pathological pathways such as cytosolic and mitochondrial calcium overload, endoplasmic reticulum stress through proteasomal dysfunction and impaired autophagy function (Fisher's exact test with FDR-adjusted P<0.05; FIG. 8A). Therefore, it is assumed that these gene expression changes are a useful measure of the efficacy of the Cas13d-based approach. In comparing individual HD neuronal cultures treated with Cas13d-CAG^{EX} or Cas13d-NT to all 3 control lines treated with Cas13d-NT, it was determined that 61.2% (605 out of 988 DEGs), 77.1% (762 out of 988 DEGs) and 57.5% (568 out of 988 DEGs) of HD DEG signatures were partially reversed in the HD 109, HD 77 and HD 66 lines, respectively, treated with Cas13d-CAG^{EX}, with evident reversal defined as a fold change of 50% or more in the expression of each DEG toward wild-type (WT) levels satisfying an FDR-adjusted P<0.01. Furthermore, reversed DEGs were enriched for each HD-associated pathological pathway mentioned above, suggesting that the described gene therapy can alleviate previously reported downstream effects of mHTT (FIG. 8A). It was also observed that 48% (HD 109), 56% (HD 77) and 45% (HD 66) of the most robust HD DEGs (twofold change, P<0.00001) were partially reversed (FIGS. 3D-3F). In addition to the common 988 DEGs, it was also determined that patient-specific DEGs (FDR-adjusted P<0.0001) were also partially reversed by 76.7% (4,582 out 5,973) in HD 109 and 81.3% (4,870 out of 5,991) in HD 77 and to a lesser degree (36.2%; 1,830 out of 5,055) in HD 66 (which harbored only approximately 80% of the extent of changes in HD 77 and HD 109) by Cas13d-CAG^{EX} treatment. These results demonstrate that the gene therapy approach reduces the most prominent disease markers common in both groups and individual patients.

Additionally, neither quantification of HTT mRNA by transcript expression levels (transcripts per million) nor qPCR could detect a significant change in total HTT mRNA levels in any of the 3 control lines treated with Cas13d-CAG^{EX} compared to Cas13d-NT or left untreated (one-way ANOVA, Tukey's post hoc test, P<0.05) (FIGS. 8B-8C). To assess the potential off-target effects of Cas13d-CAG^{EX} on other transcripts with non-disease-associated CAG repeats, the expression levels of protein-coding transcripts with five or more CAG, AGC or GCA tandem repeats were examined, which could be inadvertently targeted by the CAG^{EX} gRNA. Of 92 transcripts with five or more repeats, seven statistically significant (twofold change, FDR-adjusted P<0.01) DEGs were detected (ZNF853, MN1, IRS1, MSH4, VASH2, LMX1A, ST6GALNAC5; FIG. 8D). Considering that most of these genes are involved in neurodevelopment, including transcriptional regulation of developmental genes (ZNF8585, MN1, LMX1A) and axon development (VASH2), it is uncertain how their dysregulation will affect the adult brain. However, none of these DEGs are associated with adult-onset neurological disorders. These data suggest that other CAG-containing genes may be minimally affected by the Cas13d-based gene therapy approach.

### Example 4—Therapeutic Efficacy and Safety of Cas13d-CAG^{EX} In Vivo

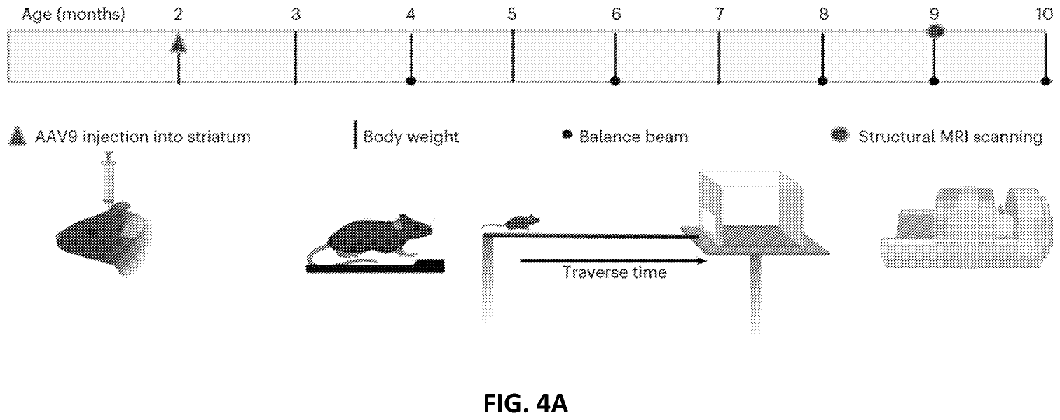

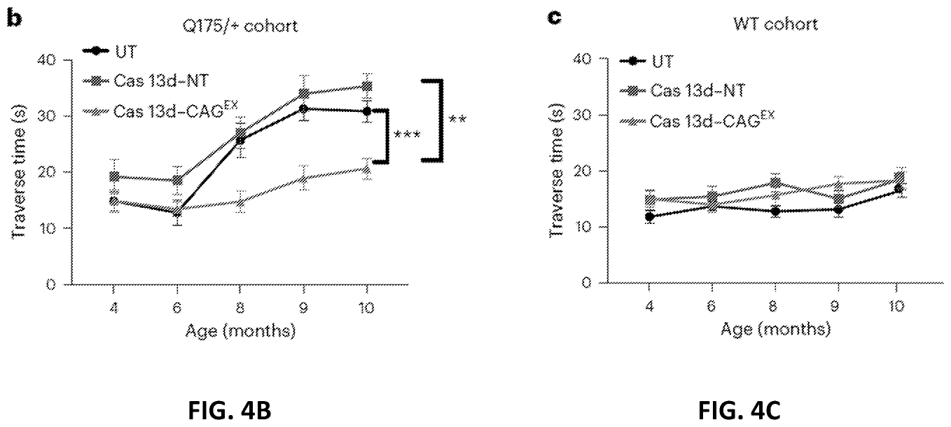

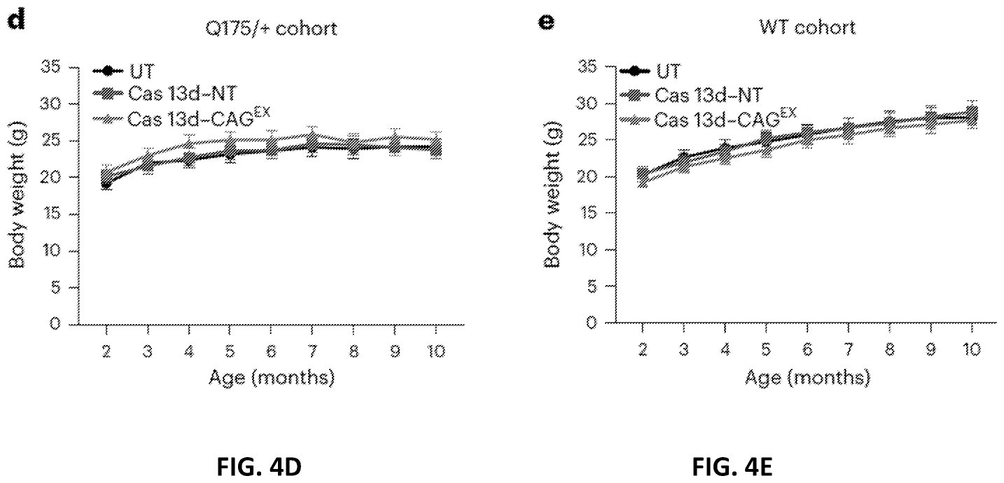

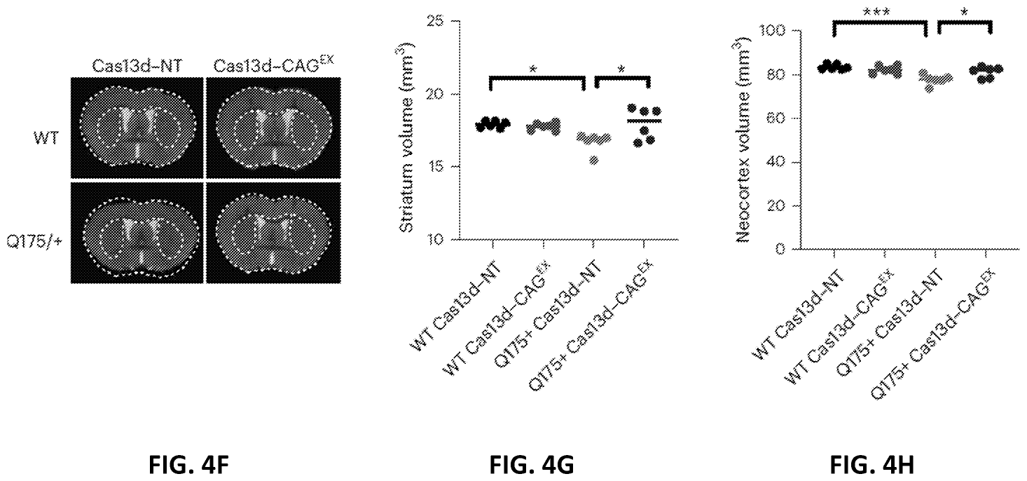

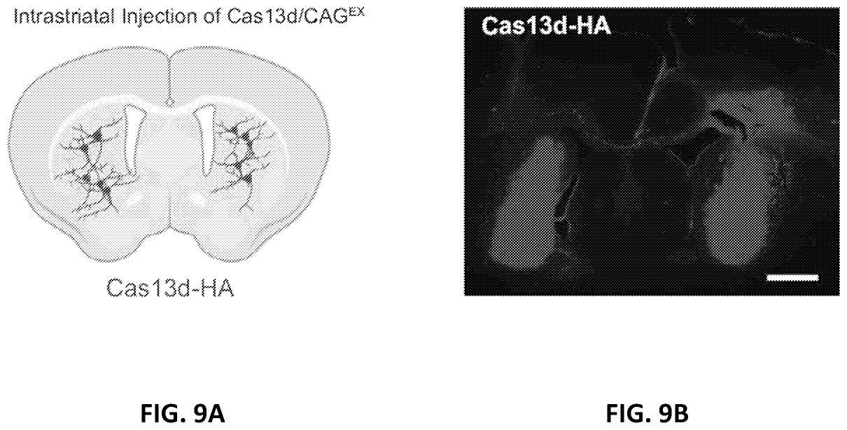

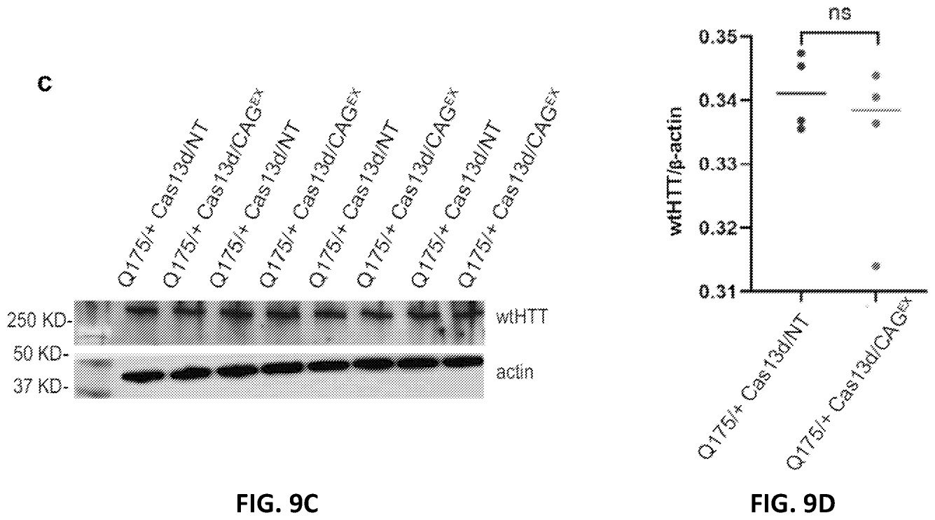

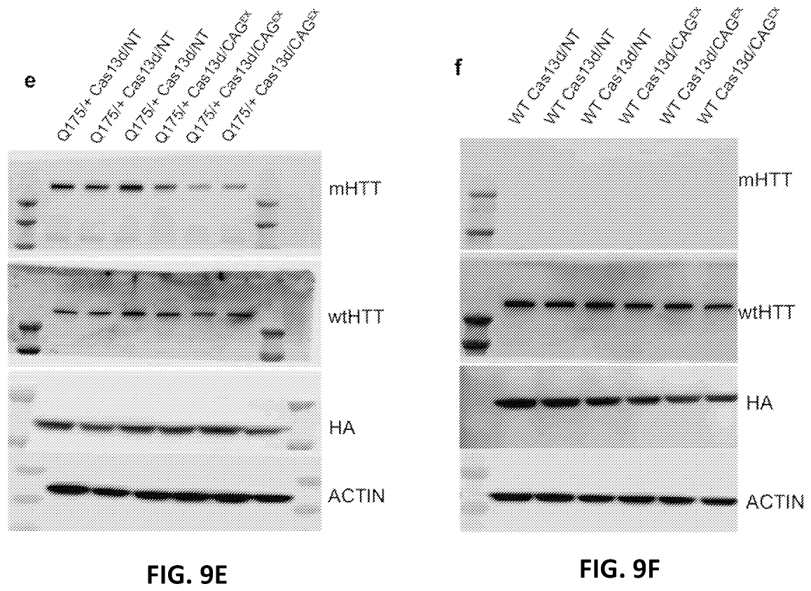

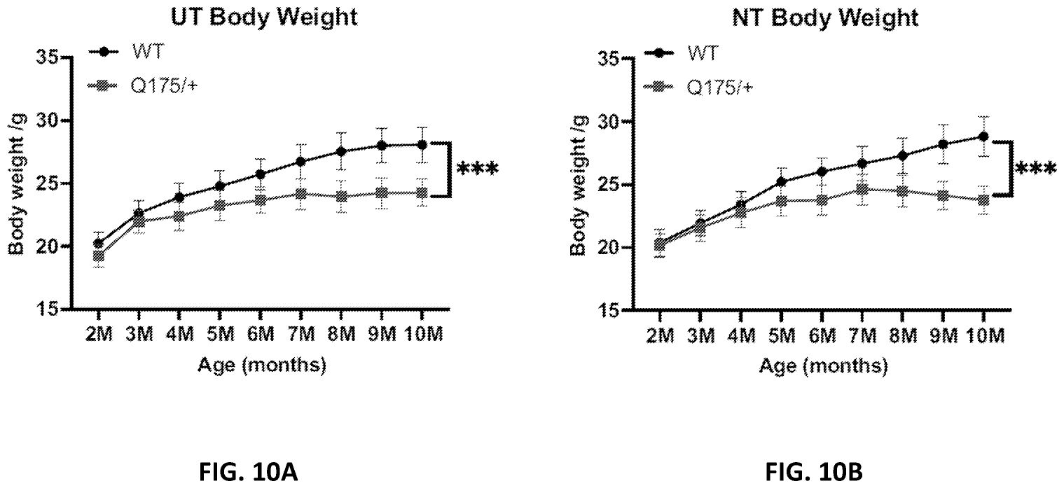

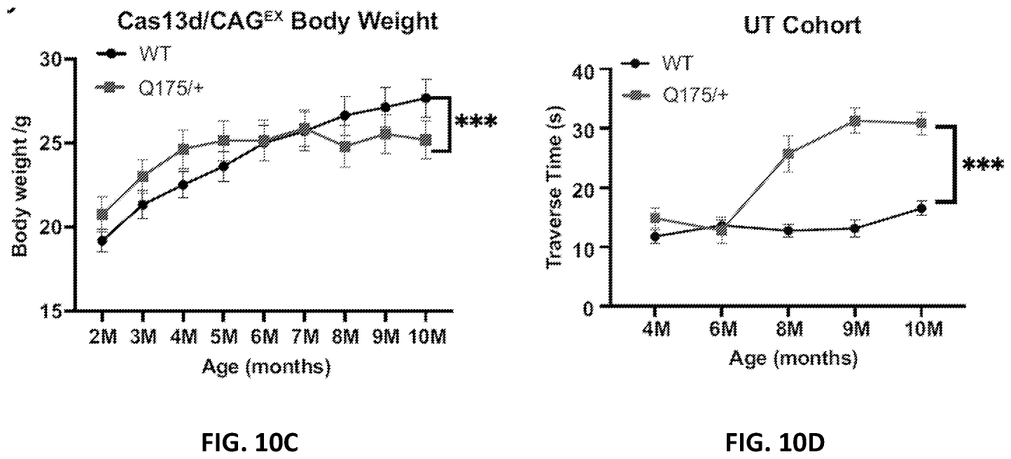

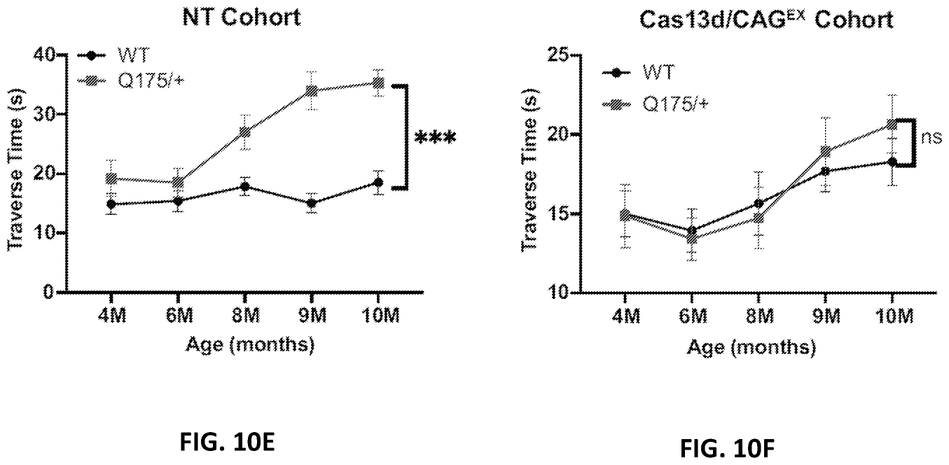

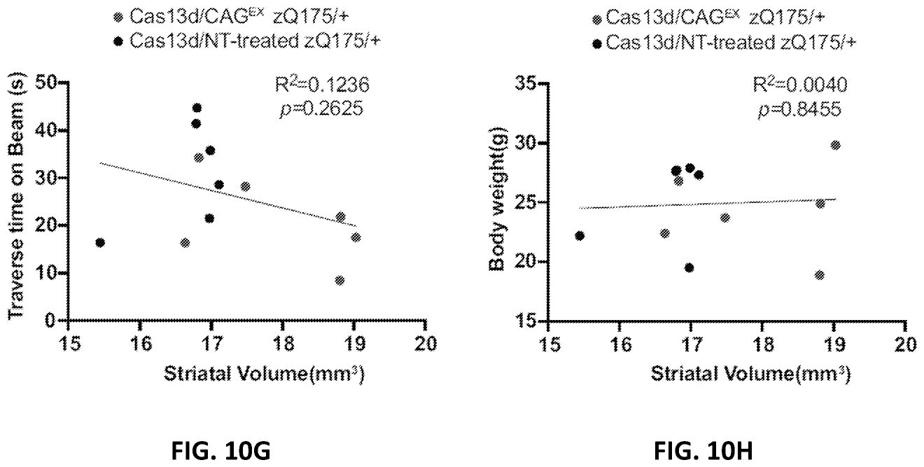

To determine if the Cas13d-based gene therapy approach can prevent or halt the progression of neurodegenerative phenotypes in vivo in zQ175/+ mice, Cas13d-CAG^{EX} or Cas13d-NT was packaged into single-stranded AAV serotype 9 (AAV9) viral vectors and conducted bilateral intrastriatal injection of Cas13d-CAG^{EX} or Cas13d-NT to an equal number of two-month-old zQ175/+HD mice and age-matched WT littermate controls. At three weeks postinjection, successful expression of both Cas13d-HA fusion constructs was observed in mouse striatum, indicated by fluorescent signals of HA protein with an anti-HA antibody (FIGS. 9A-9B). Motor function and body weight were monitored longitudinally over eight months postinjection as illustrated (FIG. 4A) and mHTT aggregates, HTT mRNA and protein levels were determined at the end of the study. The effect of treatment on motor function on a balance beam was evaluated. While zQ175/+HD mice exhibited clear motor deficits at eight months of age and after; those treated with Cas13d-CAG^{EX} showed significantly improvements in motor performance, indicated by shorter traverse times, compared with zQ175/+ mice treated with Cas13d-NT (P<0.001) or left untreated (P<0.01) (FIG. 4B and FIGS. 10D-10F). Furthermore, Cas13d-CAG^{EX} had no effect on motor function in WT mice (FIG. 4C) and did not alter body weight in zQ175/+ (FIG. 4D) or WT mice (FIG. 4E), implying that Cas13d-CAG^{EX} does not produce gross adverse effects in mice.

Next, it was examined whether Cas13d-CAG^{EX}-mediated mHTT silencing could improve neuropathology in zQ175/+ mice. Using human-translatable high-resolution structural magnetic resonance imaging (MRI), regional brain volumes were delineated accurately using an automatic segmentation, large deformation diffeomorphic metric mapping (LDDMM) including the striatum and neocortex. MRI scans of nine-month-old zQ175/+ mice were performed, when these HD mice display striatal atrophy. The behavioral results indicate that Cas13d-NT has no significant effect compared to the untreated control mice; thus, focus was put on four groups in the MRI study: WT or HD mice injected with Cas13d-NT or Cas13d-CAG^{EX}. It was demonstrated that zQ175/+ mice injected with Cas13d-NT exhibit significantly reduced striatal volume and neocortex volume compared to WT mice with the same injection, while Cas13d-CAG^{EX}-injected zQ175/+ mice have preserved striatal volume and neocortex volume compared to HD mice injected with Cas13d-NT (by group mean analysis; FIGS. 4F-4H). In addition, correlation analysis was performed between striatal volume and traverse time or body weight in zQ175/+ mice treated with either Cas13d-NT or Cas13d-CAG^{EX} and it was found that there was no significant correlation between striatal volume and traverse time in zQ175/+ mice (P=0.2625; FIG. 10G) or body weight (P=0.3755; FIG. 10H). It was noted that the brain volume data comparison is between two groups of HD mice treated with either Cas13d-CAG^{EX} or Cas13d-NT. By considering the brain volumetric measurement of individual zQ175/+ mice, the results suggest that the individual variability in striatal volume of zQ175/+ mice in each treatment group is mainly due to natural variation rather than different responders to the treatment.

### Example 5—Selective mHTT Knockdown by Cas13d-CAG^{EX} in zQ175/+ Mice

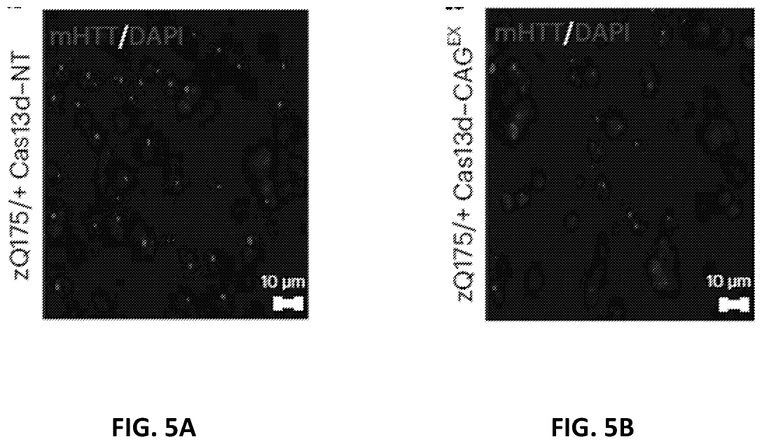

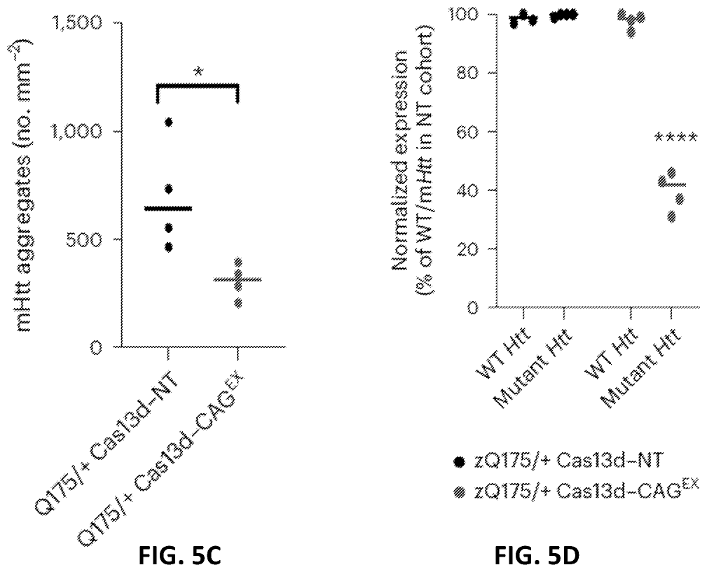

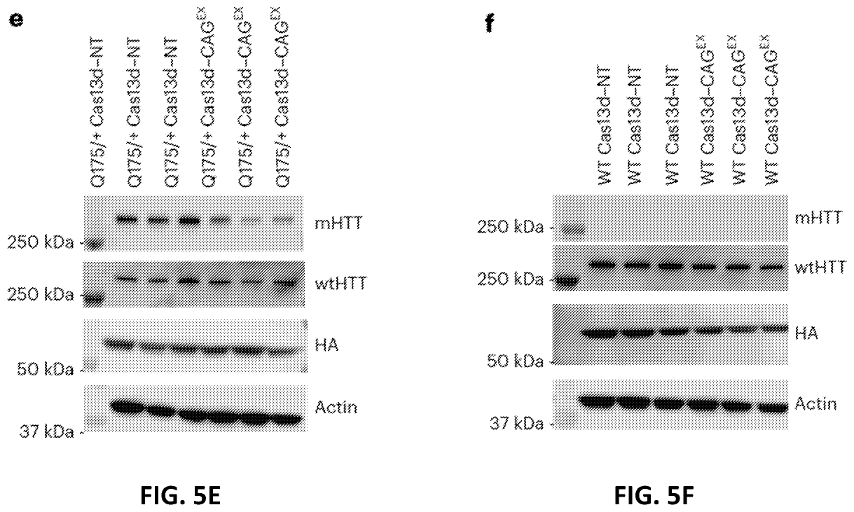

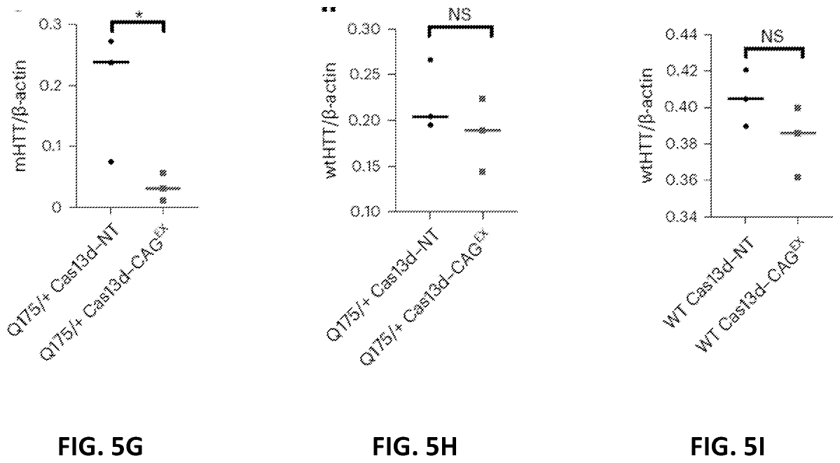

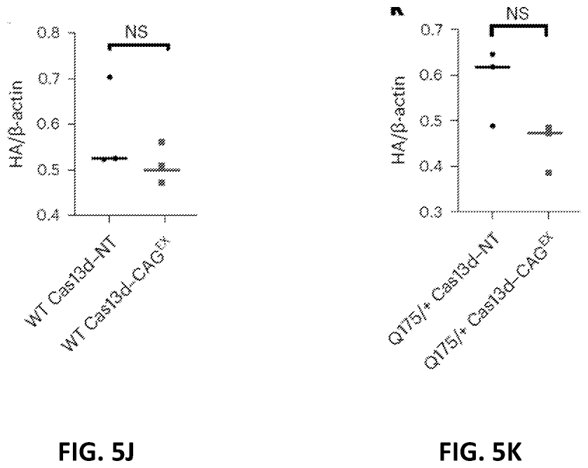

Next, the effect of Cas13d-CAG^{EX} on mHTT aggregation in zQ175/+ mouse striatum was determined. The Cas13d-CAG^{EX}-injected striatum of zQ175/+ mice harbored significantly reduced EM48+ mutant aggregates compared with those injected with Cas13d-NT (FIGS. 5A-5C), indicating that Cas13d-CAG^{EX} can effectively suppress mutant HTT aggregates in vivo. A significant reduction of mHTT HTT and protein levels was observed in the striatum of Cas13d-CAG^{EX}-injected zQ175/+ mice. WT mouse HTT mRNA and protein levels, which could be distinguished from the mutant human exon-containing HTT allele, were preserved in both zQ175/+ and WT mice treated with Cas13d-CAG^{EX} (FIGS. 5D-5F; quantified in FIGS. 5G-5K). This once again demonstrates the potential for allele-selective targeting of pathogenic HTT mRNA using Cas13d-CAG^{EX}.

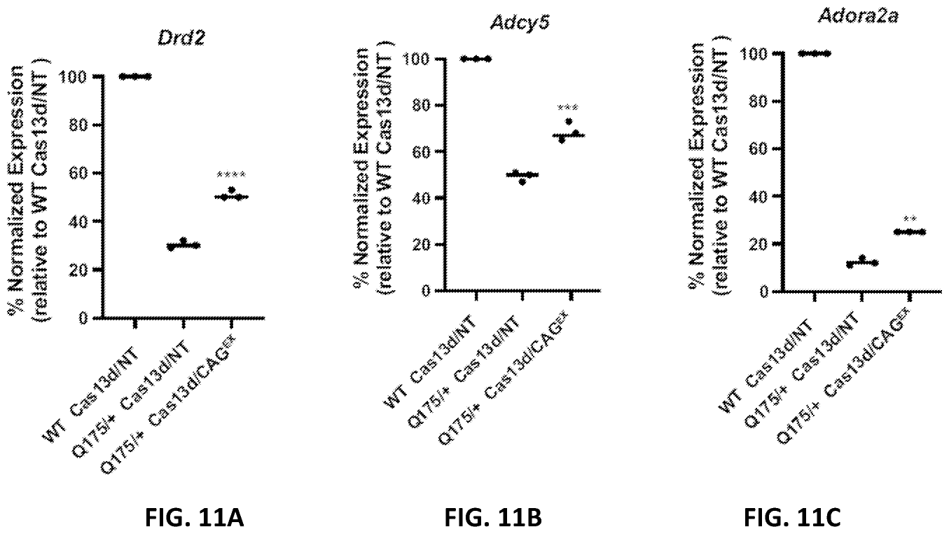

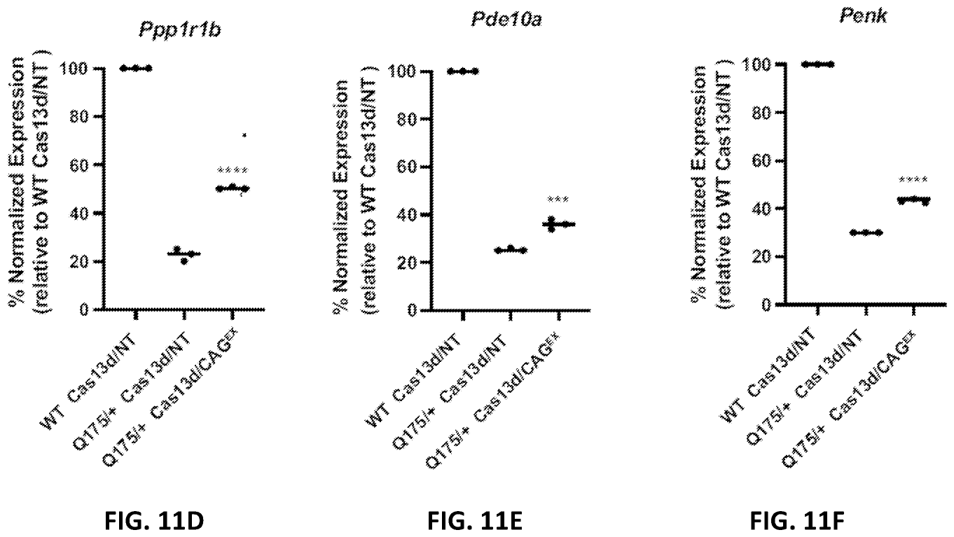

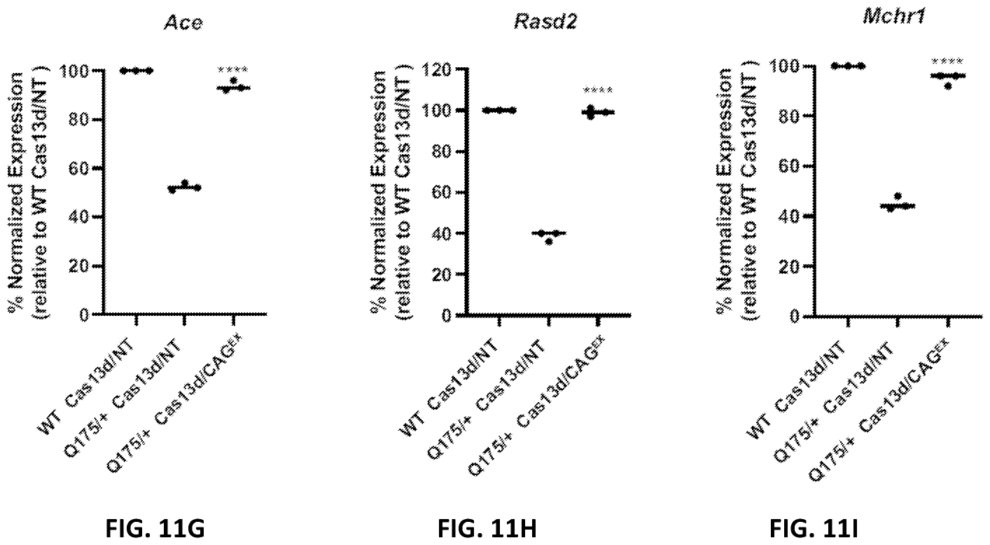

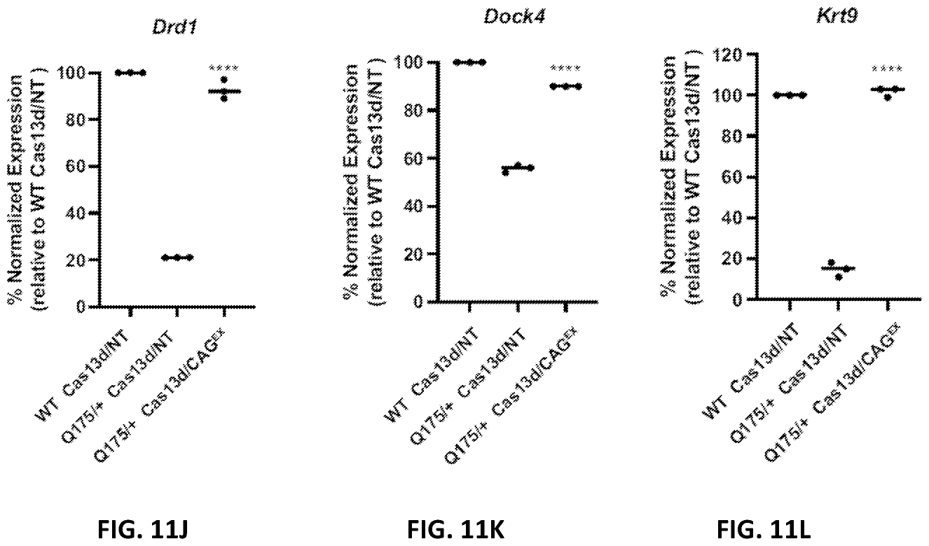

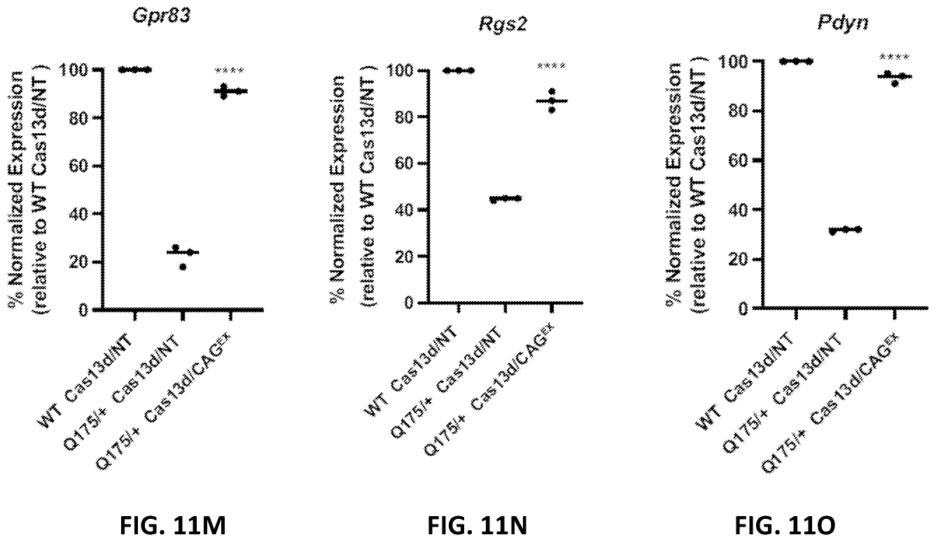

Transcriptome-wide analyses were then performed on striatal samples isolated from zQ175/+ and WT control mice treated with Cas13d-CAG^{EX} or Cas13d-NT. In doing so, widespread transcriptome dysregulation was identified caused by mHTT with a total of 2,413 downregulated and 2,368 upregulated DEGs in the Cas13d-NT-treated zQ175/+ mice compared to WT littermates (FDR-adjusted P<0.01). GO analysis showed that many of the DEGs in zQ175/+ mice are involved in ‘CAMP signaling’, ‘protein phosphorylation’ and ‘negative regulation of cell cycle transition’. These results include a large panel of established striatum-specific markers of HD (Fisher's exact test with FDR-adjusted P<0.05; FIG. 11A). Statistically significant overlaps were observed between DEGs from the Q175/+ studies and those previously reported (hypergeometric P<0.0001; FIG. 11B).

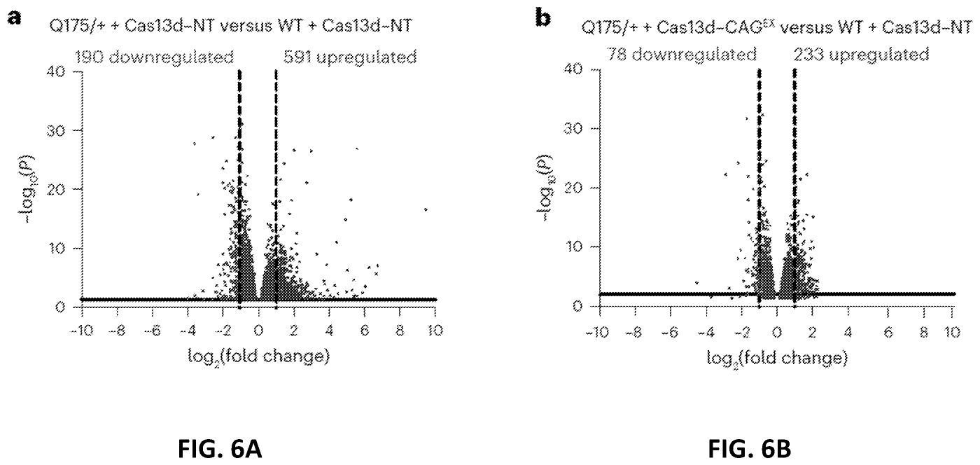

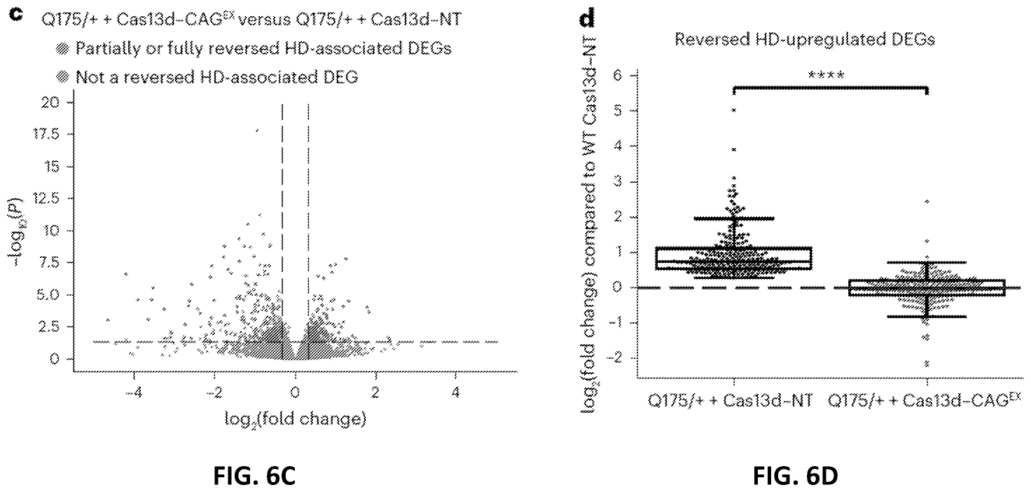

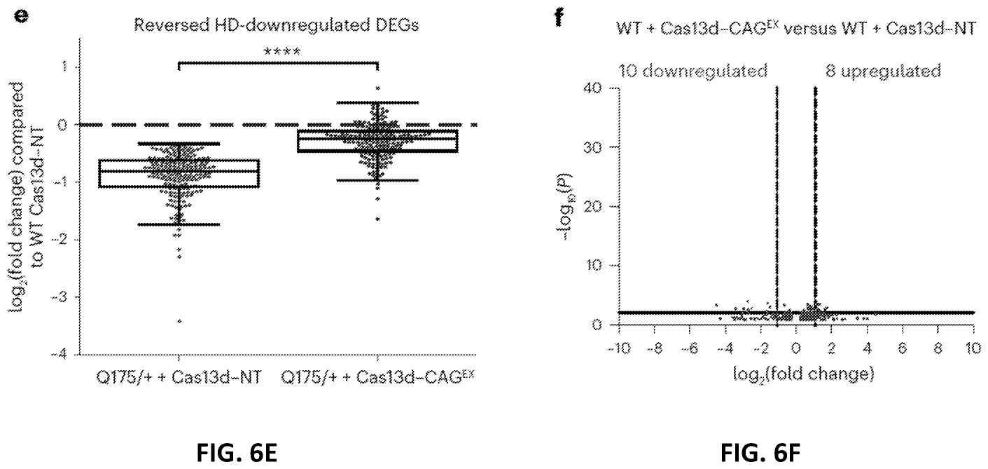

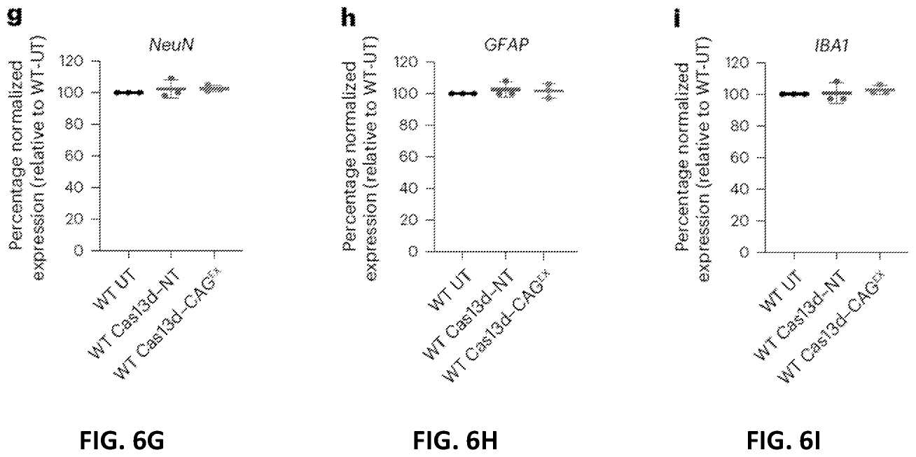

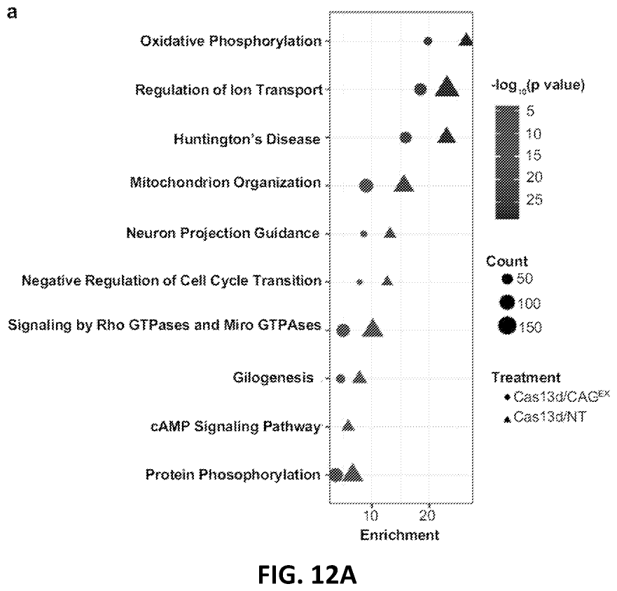

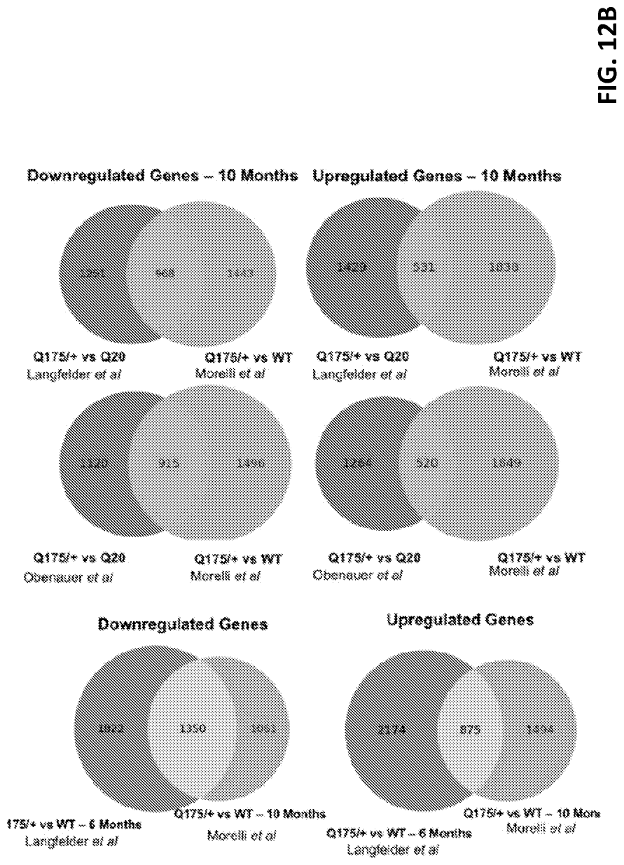

It was next determined that approximately 56% of these mHTT-mediated transcriptional changes identified in Q175/+ mice treated with Cas13d-NT (2,677 out of 4,781 DEGs) were reversed by Cas13d-CAG^{EX} treatment with a fold change of 50% or more toward WT levels (satisfying an FDR-adjusted P threshold of <0.01). It was also found that 60.2% of the most robust HD DEGs (twofold change, FDR-adjusted P<0.001) were partially to fully reversed, including most of previously reported striatum-specific HD disease markers identified in the control-treated zQ175/+ mice (59 out of 74; 79.7%; FIGS. 6A-6B and FIG. 11A-11O). The DEGs that were corrected were evenly enriched for each HD-associated pathological pathway identified in the zQ175/+ mice treated with Cas13d-NT, supporting that the approach described herein can mitigate some known downstream effects of mHTT (FIG. 12A). Furthermore, when comparing zQ175/+ mice treated with Cas13d-CAG^{EX} to ones treated with Cas13d-NT, 66.5% (189 out of 284) of upregulated DEGs and 54.0% (238/441) of downregulated DEGs partially to fully reversed to resemble WT levels, that is, if they exhibited a significant reduction in difference between their zQ175/+ and WT levels (FDR-adjusted P<0.01, difference of more than 20% and depicted in FIG. 6C). Some genes achieved almost complete rescue (P<10-4, Wilcoxon two-sample paired signed-rank test; FIGS. 6D-6E).

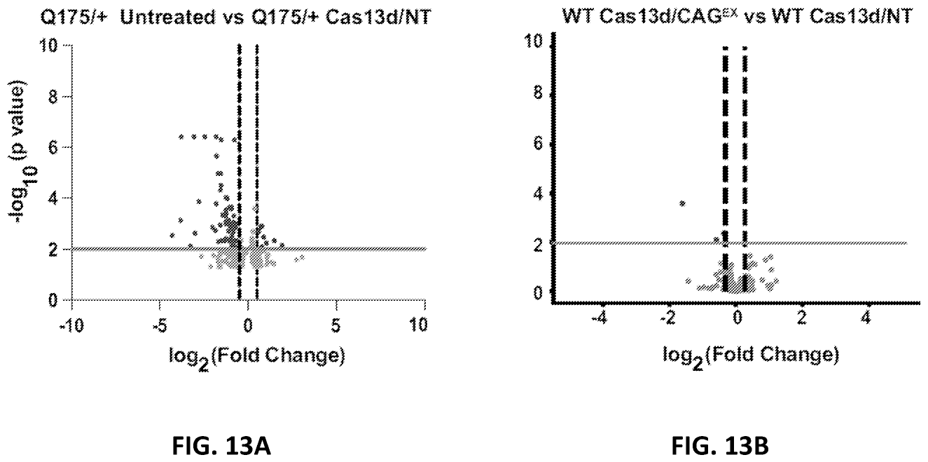

The safety profile of Cas13d-CAG^{EX} was next evaluated based on our RNA-seq data. It was observed that approximately 80 genes are differentially expressed when comparing Cas13d-NT-treated and untreated zQ175/+ mice, which may be a result of injection, the AAV9 viral delivery vector, Cas13d, the gRNA or a combination of these factors (FIG. 13A). Nevertheless, when comparing WT mice treated with Cas13d-CAG^{EX} or Cas13d-NT, only 18 were found to show statistically significant (FDR-adjusted P<0.01) changes in gene expression (FIG. 6F). The expression levels of protein-coding transcripts with five or more non-disease-associated CAG, AGC or GCA tandem repeats were then examined, which could be inadvertently targeted by the CAG^{EX} gRNA. Of 344 transcripts with 5 or more repeats, only 3 significant DEGs (FDR-adjusted P<0.01) were detected (FIG. 13B). Furthermore, qPCR also confirmed that Cas13d-CAG^{EX} did not disrupt CNS-specific markers of immunogenicity including neuronal nuclei (NeuN), glial fibrillary acidic protein (GFAP) and ionized calcium binding adaptor molecule 1 (IBA1) (FIGS. 6G-6I). Taken together, these results support the efficacy and safety of Cas13d-CAG^{EX} in HD preclinical models.

What is claimed is:

1. A method of treating Huntington's disease in a subject, the method comprising administering to the subject a therapeutically effective amount of a pharmaceutical composition comprising:
(a) a guide RNA (gRNA), wherein the guide RNA comprises a repeat sequence complementary to a target CAG-expansion RNA, and wherein the gRNA allele-selectively targets the target CAG-expansion RNA; and
(b) a CRISPR-associated protein or a nucleic acid sequence encoding the CRISPR-associated protein, wherein the CRISPR-associated protein comprises a Cas13d polypeptide.

2. The method of claim 1, wherein the repeat sequence of the guide RNA comprises GTC, TCG, or CGT.

3. The method of claim 1 or 2, wherein the gRNA comprises a sequence selected from the group consisting of SEQ ID NO: 1, SEQ ID NO: 2, or SEQ ID NO: 3.

4. The method of any one of claims 1-3, wherein the gRNA further comprises a U6 promoter sequence.

5. The method of any one of claims 1-4, wherein the target CAG-expansion RNA comprises more than 30 copies of a CAG repeat sequence.

6. The method of any one of claims 1-4, wherein the target CAG-expansion RNA comprises between 31 and 60 copies of a CAG repeat sequence.

7. The method of any one of claims 1-4, wherein the target CAG-expansion RNA comprises more than 60 copies of a CAG repeat sequence.

8. The method of any one of claims 1-4, wherein the target CAG-expansion RNA comprises more than 100 copies of a CAG repeat sequence.

9. The method of any one of claims 1-8, wherein the target CAG-expansion RNA comprises a mutant huntingtin gene (HTT).

10. The method of any one of claims 1-9, wherein the Cas13d is Ruminococcus Flavefaciens XPD3002 (Rfx) Cas13d.

11. The method of any one of claims 1-10, wherein the Cas13d is tagged with a human influenza hemagglutinin (HA) epitope.

12. The method of any one of claims 1-11, wherein the nucleic acid sequence encoding the Cas 13d polypeptide further comprises an RNA polymerase II promoter sequence or an RNA polymerase III promoter sequence.

13. The method of claim 12, wherein the RNA polymerase III promoter sequence is an EF1a promoter sequence.

14. The method of any one of claims 1-13, wherein the pharmaceutical composition comprises a vector, wherein the vector comprises at least one of (a) gRNA and (b) CRISPR-associated protein.

15. The method of claim 14, wherein the vector comprises both (a) and (b).

16. The method of claim 15, wherein the vector is a viral vector.

17. The method of claim 16, wherein the viral vector is an adeno-associated viral vector (AAV), lentiviral vector, or an adenoviral vector.

18. The method of any one of claims 1-17, wherein the subject is under 18 years old.

19. The method of any one of claims 1-17, wherein the subject is older than 18 years old.

20. The method of any one of claims 1-19, wherein the administration of the pharmaceutical composition comprises intrastriatal administration.

21. The method of any one of claims 1-20, wherein the administration of the pharmaceutical composition reduces mRNA and/or protein expression of the target CAG-expansion RNA.

22. A recombinant allele-selective expression system for CRISPR/Cas-directed RNA targeting of a target CAG-expansion RNA comprising:
(a) a guide RNA, wherein the guide RNA comprises a repeat sequence complementary to the target CAG-expansion RNA; and
(b) a CRISPR-associated protein or a nucleic acid sequence encoding the CRISPR-associated protein, wherein the CRISPR-associated protein comprises a Cas13d polypeptide.

23. The recombinant allele-selective expression system of claim 22, wherein the repeat sequence of the guide RNA comprises GTC, TCG, or CGT.

24. The recombinant allele-selective expression system of claim 22 or 23, wherein the guide RNA comprises a sequence selected from the group consisting of SEQ ID NO: 1, SEQ ID NO: 2, or SEQ ID NO: 3.

25. The recombinant allele-selective expression system of any one of claims 22-24, wherein the gRNA further comprises a U6 promoter sequence.

26. The recombinant allele-selective expression system of any one of claims 22-25, wherein the target CAG-expansion RNA comprises more than 30 copies of a CAG repeat sequence.

27. The recombinant allele-selective expression system of any one of claims 22-26, wherein the target CAG-expansion RNA comprises between 31 and 60 copies of a CAG repeat sequence.

28. The recombinant allele-selective expression system of any one of claims 22-26, wherein the target CAG-expansion RNA comprises more than 60 copies of a CAG repeat sequence.

29. The recombinant allele-selective expression system of any one of claims 22-26, wherein the target CAG-expansion RNA comprises more than 100 copies of a CAG repeat sequence.

30. The recombinant allele-selective expression system of any one of claims 22-29, wherein the target CAG-expansion RNA comprises a mutant huntingtin gene (HTT).

31. The recombinant allele-selective expression system of any one of claims 22-29 wherein the Cas13d is Ruminococcus Flavefaciens XPD3002 (Rfx) Cas13d.

32. The recombinant allele-selective expression system of any one of claims 22-31, wherein the Cas13d is tagged with an HA epitope.

33. The recombinant allele-selective expression system of any one of claims 22-32, wherein the nucleic acid sequence encoding the Cas13d polypeptide further comprises an RNA polymerase II promoter sequence or an RNA polymerase III promoter sequence.

34. The recombinant allele-selective expression system of claim 33, wherein the RNA polymerase III promoter sequence is an EF1a promoter sequence.

35. The recombinant allele-selective expression system of any one of claims 22-34, wherein the recombinant expression system is delivered into a cell.

36. The recombinant allele-selective expression system of claim 35, wherein the cell is a mammalian cell.

37. The recombinant allele-selective expression system of claim 35, wherein the cell is derived from a subject diagnosed as having Huntington's disease.

38. The recombinant allele-selective expression system of any one of claims 22-37, wherein the recombinant expression system is comprised in a vector.

39. The recombinant allele-selective expression system of claim 38, wherein (a) and (b) are comprised within a same vector.

40. The recombinant allele-selective expression system of claim 38, wherein the vector is a viral vector.

41. The recombinant allele-selective expression system of claim 40, wherein the viral vector is an adeno-associated viral vector (AAV), lentiviral vector, or an adenoviral vector.

42. A vector comprising a nucleic acid encoding (i) a guide RNA, wherein the guide RNA comprises a repeat sequence complementary to a target CAG-expansion RNA, and wherein the gRNA allele-selectively targets the target CAG-expansion RNA; and (ii) a CRISPR-associated protein, wherein the CRISPR-associated protein comprises a Cas13d polypeptide.

43. The vector of claim 42, wherein the repeat sequence of the guide RNA comprises GTC, TCG, or CGT.

44. The vector of claim 42 or 43, wherein the gRNA comprises a sequence selected from the group consisting of SEQ ID NO: 1, SEQ ID NO: 2, or SEQ ID NO: 3.

45. The vector of any one of claims 42-44, wherein the gRNA further comprises a U6 promoter sequence.

46. The vector of any one of claims 42-45, wherein the target CAG-expansion RNA comprises more than 30 copies of a CAG repeat sequence.

47. The vector of any one of claims 42-45, wherein the target CAG-expansion RNA comprises between 31 and 60 copies of a CAG repeat sequence.

48. The vector of any one of claims 42-45, wherein the target CAG-expansion RNA comprises more than 60 copies of a CAG repeat sequence.

49. The vector of any one of claims 42-45, wherein the target CAG-expansion RNA comprises more than 100 copies of a CAG repeat sequence.

50. The vector of any one of claims 42-49, wherein the target CAG-expansion RNA comprises a mutant huntingtin gene (HTT).

51. The vector of any one of claims 42-50, wherein the Cas13d is Ruminococcus Flavefaciens XPD3002 (Rfx) Cas13d.

52. The vector of any one of claims 42-51, wherein the Cas13d is tagged with an HA epitope.

53. The vector of any one of claims 42-52, wherein the nucleic acid sequence encoding the Cas 13d polypeptide further comprises an RNA polymerase II promoter sequence or an RNA polymerase III promoter sequence.

54. The vector of claim 53, wherein the RNA polymerase III promoter sequence is an EF1a promoter sequence.

55. The vector of any one of claims 42-54, wherein the vector is delivered into a cell.

56. The vector of claim 55, wherein the cell is a mammalian cell.

57. The vector of claim 55, wherein the cell is derived from a subject diagnosed as having Huntington's disease.

58. The vector of any one of claims 42-57, wherein the vector is a viral vector.

59. The vector of claim 58, wherein the viral vector is an adeno-associated viral vector (AAV), lentiviral vector, or an adenoviral vector.

60. A cell comprising a recombinant allele-selective expression system of any one of claims 22-41 or a vector of any one of claims 42-59.

61. A pharmaceutical composition comprising the cell of claim 60 and a pharmaceutically acceptable carrier.


SEQUENCE LISTING (3 sequences)

SEQ ID NO: 1 - DNA, 21 bp, synthetic construct
  source 1..21: mol_type = other DNA
gtcgtcgtcgtcgtcgtcgtc

SEQ ID NO: 2 - DNA, 21 bp, synthetic construct
  source 1..21: mol_type = other DNA
tcgtcgtcgtcgtcgtcgtcg

SEQ ID NO: 3 - DNA, 21 bp, synthetic construct
  source 1..21: mol_type = other DNA
cgtcgtcgtcgtcgtcgtcgt